SEQUENCE LISTING (28 sequences)

SEQ ID NO: 1 - DNA, 23 bp, Artificial Sequence
  Description of Artificial Sequence Cannabis sativa primer
gtcagaaagcgaagacctttaga

SEQ ID NO: 2 - DNA, 19 bp, Artificial Sequence
  Description of Artificial Sequence Cannabis sativa primer
gtaaagacaggcagccatc

SEQ ID NO: 3 - DNA, 36 bp, Artificial Sequence
  Description of Artificial Sequence Cannabis sativa primer
tcaattaatgcttatagcccatatgttttctactac

SEQ ID NO: 4 - DNA, 33 bp, Artificial Sequence
  Description of Artificial Sequence Cannabis sativa primer
agaacaagaagaaacaaagtattcctgaagttg

SEQ ID NO: 5 - DNA, 28 bp, Artificial Sequence
  Description of Artificial Sequence Cannabis sativa primer
tttgccattgatttcctcctcctcatac

SEQ ID NO: 6 - DNA, 23 bp, Artificial Sequence
  Description of Artificial Sequence Cannabis sativa primer
agatcccaagcttcccgggtacc

SEQ ID NO: 7 - DNA, 36 bp, Artificial Sequence
  Description of Artificial Sequence Cannabis sativa primer
ctacaaagattcccacactcaataatgcaaatacaa

SEQ ID NO: 8 - DNA, 31 bp, Artificial Sequence
  Description of Artificial Sequence Cannabis sativa primer
agtaaggatttggttttcggctttcgttctt

SEQ ID NO: 9 - DNA, 35 bp, Artificial Sequence
  Description of Artificial Sequence Cannabis sativa primer
caaaaattcatacataaggcacgaagagatagaca

SEQ ID NO: 10 - DNA, 27 bp, Artificial Sequence
  Description of Artificial Sequence Cannabis sativa primer
tttatacagtcctatcgccgggtccaa

SEQ ID NO: 11 - DNA, 37 bp, Artificial Sequence
  Description of Artificial Sequence Cannabis sativa primer
ttttcataattgtttgcaaaataatctttctctagaa

SEQ ID NO: 12 - DNA, 33 bp, Artificial Sequence
  Description of Artificial Sequence Cannabis sativa primer
gttgtggtcatggtgggaagtataattttaata

SEQ ID NO: 13 - DNA, 23 bp, Artificial Sequence
  Description of Artificial Sequence Cannabis sativa primer
caaagagtgtatcgaaacctgtc

SEQ ID NO: 14 - DNA, 25 bp, Artificial Sequence
  Description of Artificial Sequence Cannabis sativa primer
gtactaatacagacgatgtggtggg

SEQ ID NO: 15 - DNA, 36 bp, Artificial Sequence
  Description of Artificial Sequence Cannabis sativa primer
atagtaatatgtccaacaaaagcaaagaaagaaaaa

SEQ ID NO: 16 - DNA, 25 bp, Artificial Sequence
  Description of Artificial Sequence Cannabis sativa primer
caagtgtttcatgtgattgggccac

SEQ ID NO: 17 - DNA, 27 bp, Artificial Sequence
  Description of Artificial Sequence Cannabis sativa primer
agacgtggcatatgcgctgttccttca

SEQ ID NO: 18 - DNA, 28 bp, Artificial Sequence
  Description of Artificial Sequence Cannabis sativa primer
gcatacccattagtgaacggccatcggc

SEQ ID NO: 19 - DNA, 22 bp, Artificial Sequence
  Description of Artificial Sequence Cannabis sativa primer
ttccgacaccggcgacgcactc

SEQ ID NO: 20 - DNA, 35 bp, Artificial Sequence
  Description of Artificial Sequence Cannabis sativa primer
ttctttcccatatttttcatcatctcttcgtcgaa

SEQ ID NO: 21 - DNA, 23 bp, Artificial Sequence
  Description of Artificial Sequence Cannabis sativa primer
ggtaagttgatacattccttccc

SEQ ID NO: 22 - DNA, 21 bp, Artificial Sequence
  Description of Artificial Sequence Cannabis sativa primer
caagtagcctttggtcactgc

SEQ ID NO: 23 - DNA, 19 bp, Artificial Sequence
  Description of Artificial Sequence Cannabis sativa primer
ggatcagcggcaacaacaa

SEQ ID NO: 24 - DNA, 25 bp, Artificial Sequence
  Description of Artificial Sequence Cannabis sativa primer
tgttatgtctgctctacccagtttt

SEQ ID NO: 25 - DNA, 25 bp, Artificial Sequence
  Description of Artificial Sequence Cannabis sativa primer
gactcagcatattaaagcagaaact

SEQ ID NO: 26 - DNA, 25 bp, Artificial Sequence
  Description of Artificial Sequence Cannabis sativa primer
gtttacatattccacttgtttgtga

SEQ ID NO: 27 - DNA, 18 bp, Artificial Sequence
  Description of Artificial Sequence Cannabis sativa primer
gcagtggatgtgtcgggt

SEQ ID NO: 28 - DNA, 23 bp, Artificial Sequence
  Description of Artificial Sequence Cannabis sativa primer
tttgtgcctgacctaatcctcta

(19) United States
(12) Patent Application Publication
(10) Pub. No.: US 2006/0035236 A1
(43) Pub. Date: Feb. 16, 2006

(54) DNA fingerprinting for Cannabis sativa (marijuana) using short tandem repeat (STR) markers

(76) Inventors: Paul S. Keim, Flagstaff, AZ (US); Kristen Zinnamon, Flagstaff, AZ (US)

Correspondence Address: QUARLES & BRADY LLP, RENAISSANCE ONE, TWO NORTH CENTRAL AVENUE, PHOENIX, AZ, 85004-2391, US

(21) Appl. No.: 10/624,217

(22) Filed: Jul. 21, 2003

Related U.S. Application Data
(60) Provisional application No. 60/397,179, filed on Jul. 19, 2002.

(51) Int. Cl. C12Q 1/68; C07H 21/04

(52) U.S. Cl. 435/6; 536/23.6

(57) ABSTRACT

Multiplex methods for discriminating among Cannabis sativa L. plants are disclosed. Eight STR loci have been identified from genomic sequences of Cannabis sativa L. plants and primer pairs and cocktails suitable for amplifying the STR by multiplex are disclosed. Polymorphisms at these loci were used to resolve genotypes into distinct groups. Kits are provided for use with multiplex instruments to identify DNA in a plant sample. The typing scheme is useful for the forensic identification of marijuana and for linking a marijuana sample to its plant source.

### CLAIM TO DOMESTIC PRIORITY

This application claims benefit of priority to U.S. Provisional application Ser. No. 60/397,179, entitled “DNA Fingerprinting For Cannabis sativa (Marijuana) Using Short Tandem Repeat (STR) Markers” filed Jul. 19, 2002, by Paul S. Keim et al., and is herein incorporated by reference in its entirety.

### FIELD OF THE INVENTION

This invention concerns the molecular analysis of Cannabis sativa L. (marijuana) and more specifically provides primer cocktails for multiplex analysis of DNA from purported Cannabis sativa L. samples to allow forensic identification and tracking of a leaf sample to its plant source.

### BACKGROUND

Cannabis sativa L. is one of the oldest crops known to man (Siniscalco Gigliano 2001). Despite its long historical relationship with human civilization, still relatively little is known about the genetic composition of this plant. However, recently many studies have tried to examine the molecular characteristics of Cannabis in order to distinguish hemp (fiber) varieties from marijuana (drug) varieties (Gilmore et al. 2003).

The historical and intimate association between Cannabis sativa L. (marijuana) and man has no doubt contributed to this plant's many varieties and uses [1,2]. It is commonly believed that humans introduced C. sativa to the Americas in 1545; but before its worldwide introduction, it likely originated and was native to central Asia [3,4]. From even the earliest accounts, man has utilized virtually all parts of the plant for a multitude of purposes, the two most common uses being harvesting the plant for its fiber and drug qualities [5]. The flowers and leaves of the plant are harvested for the chemical resin, delta-9-tetrahydrocannabinol (THC), which when ingested, produces the psychoactive effects that humans experience [6].

A common problem for law enforcement agencies is the correct identification and suppression of illegal growing operations. The forensic community has made significant progress in developing molecular identification techniques for Cannabis [7-11]. Virtually all of these experiments have focused on molecular identification methods which exclusively amplify Cannabis DNA, enabling forensic investigators to move away from conventional chemical identification tests such as GC-MS, HPLC and histological microscopy. Despite these advances, tests that are capable of individualizing marijuana plants and discriminating between varieties were not available, until recently [12,13]. These kinds of tests are necessary to facilitate the identification and suppression of growing operations by forensic investigators.

Both Gilmore [12] and Hsieh [13] have investigated the potential utility of short tandem repeat (STR) markers for distinguishing and individualizing Cannabis plants. Short tandem repeats: (STRs), simple sequence repeats (SSRs), or microsatellites all describe a single type of DNA profiling technology that is useful for providing genetic information about individuals within and among populations. STR genetic markers selectively amplify hypervariable regions of DNA and, when run on gels, generate fluorescent banding patterns that can be used as unique genetic identifiers. Each STR marker is made up of a single DNA sequence, no more than six base pairs long, that is repeated in tandem and individual loci have length polymorphisms in the repeat array [14]. STR markers are useful in forensic investigations because they are polymerase chain reaction (PCR) based and are capable of amplifying small amounts of fairly degraded DNA, which is commonly the condition of biological samples from crime scenes [14]. Additionally, STR markers are desirable because they are a co-dominant marker system and they provide information about the heterozygosity of individual plants.

Methods and means for reliable and fast genetic analysis of STR markers in Cannabis sativa L. have been sought. These analyses would identify purported marijuana samples and would provide a useful forensic tool for linking the source of sample to its plant of origin.

It is an object of this invention to provide methods and means for STR, typing in Cannabis to aid forensic investigators in: (i) linking personal possessions of marijuana to plants at the person's residence, (ii) identifying clonally propagated plants as having matching genotypic profiles, and (iii) tracking the distribution patterns of clonally propagated plants within residential areas.

### SUMMARY

The present invention discloses methods and means for detecting and identifying Cannabis sativa L. species by short tandem repeat (STR) analysis multiplex genotyping system of STR identified within the genome of Cannabis sativa L. STR in the Cannabis sativa L. genome are amplified using labeled primers in multiplexed PCRs and electrophoretically separated on polyacrylamide gels for analysis.

STR loci located throughout the Cannabis sativa L. genome have been identified. Isolated nucleic acids having the sequence of STR identified in Cannabis sativa L. are presented. In an important aspect of the present invention nucleic acids comprising at least 12, 15, 18 or total consecutive nucleotides of a nucleotide sequence selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID 20; SEQ ID 21; SEQ ID 22; SEQ ID 23; SEQ ID 24; SEQ ID 25; SEQ ID 26; SEQ ID 27; SEQ ID 28; and sequences complementary thereto are presented.

In certain preferred embodiments of the invention, these nucleic acids are immobilized on a solid surface and are useful, for example, in the detection of a Cannabis sativa L. sample in an assay employing probes, including, but not limited to, a nano-detection device.

In another important aspect of the invention, primer pairs comprising a forward and a reverse primer are presented for amplification of STR located in DNA from a Cannabis sativa L. species. Primer pairs suitable for PCR amplification of STR, by multiplex, may be selected from the group consisting of SEQ ID NO: 1 and 2; SEQ ID NO: 3 and 4; SEQ ID NO: 5 and 6; SEQ ID NO: 7 and 8; SEQ ID NO: 9 and 10; SEQ ID NO: 11 and 12; SEQ ID NO: 13 and 14; SEQ ID NO: 15 and 16; and SEQ ID NO: 17 and 18; SEQ ID NO: 19 and 20; SEQ ID NO: 21 and 22; SEQ ID NO: 23 and 24; SEQ ID NO: 25 and 26; and SEQ ID NO: 27 and 28.

Combinations of the isolated nucleic acids or primer pairs described herein as “cocktails” are provided for amplification of the STR markers by multiplex. Certain preferred primer pairs have, in addition, an observable group whereby amplified product may be detected. Such groups may be, for example, a fluorescent group or a radioactive group.

In another important aspect of the invention, a method for detecting a Cannabis sativa L. species in a sample from a plant, preferably a leaf or flower sample, is presented. The method comprises the steps of:
i. obtaining DNA from the sample,
ii. amplifying a STR marker loci in said DNA with a multiplex cocktail selected from the group of primer pairs to form amplification products of various sizes and labels; and
iii. separating amplification products by size and primer label;
iv. scoring the results of said separation
v. comparing said scored results to results of analysis of DNA from a known species.

In yet another important aspect of the invention methods for linking a marijuana sample to a plant source are presented. The method comprises the steps of:
i. determining the identity of DNA in said sample by the present method
ii. determining the identity of DNA in a sample from a plant by the present method; and
iii. comparing the identities of both samples to determine similarities.

In another important aspect of the invention, multiplex methods are presented for observing polymorphisms at STR loci in DNA from more than one Cannabis sativa L. species to resolve unique genotypes between the species and to allow linking of the sample to its plant of origin. These multiplex methods provide a convenient and rapid method for genetic discrimination in Cannabis sativa L. and, for forensic purposes, provides information necessary to track the source of a purported marijuana sample. Cocktails provided herein are preferably used for amplifying STR in the multiplex methods.

In yet another important aspect of the invention, kits are herein provided for use with commercially available PCR instruments to detect a strain of Cannabis sativa L. species. The kits comprise one or more primer pairs suitable for amplifying STR in DNA in a sample of said species by PCR. Preferably the kits comprise primer pairs having SEQ ID NOS: 1-28. Most preferably kits are provided for multiplexing-DNA in a sample. These kits comprise primer pair sets, i.e., cocktails, selected from the group of primer pairs.

The kits may further comprise nucleic acids, enzymes, tag polymerase, for example, salts and buffers suitable for causing amplification by PCR, by multiplex; The kits also comprise preferably a positive control. In certain preferred embodiments of the kit the primers comprise a label whereby amplified STR may be detected. In other preferred embodiments of the kit, labeled nucleic acids are provided. Observable labels are preferably fluorescent molecules or radionucleotides. The kits may also comprise suitable containers and bottles for housing these reagents and or convenient use.

### DETAILS

Multiplex methods are presented for rapid genotyping of Cannabis sativa L. STR markers described herein provide discriminatory power that enhances the ability of present methods to determine rapidly molecular relationships of Cannabis sativa L. samples. A C. sativa STR database has been generated by multiplexing 295 samples and eight STR markers. This database illustrates that STR genetic markers in C. sativa are both hypervariable and capable of discriminating among individual plants.

This multiplex typing system is a PCR-based method for genotyping Cannabis sativa L. using eight STR loci identified in the present invention. This PCR-based typing system has advantages not present in other PCR-systems: rapid turnaround, amplification with crudely isolated or minute amounts, of DNA. The rapid typing system using eight. STR loci has been used to analyze a collection of a 295 samples to detect genotypic differences between individual C. sativa plants. Over 90% of the samples had unique multilocus genotypic profiles and some of the samples with matching profiles were known to be duplicate samples. Although the heterozygosity values detected within this system are fairly low compared to other studies of STRs in plants [12,18], this may be indicative of the selective breeding practices within drug varieties of C. sativa plants. It is known that certain drug qualities such as THC content are selectively bred for within this plant [24] and therefore, this system may be detecting some of these highly inbred genotypes. Additional markers, [12,13] would increase the observed heterozygosity values and enhance the power of an STR profiling system for C. sativa.

Tri- and tetranucleotide repeat motifs were isolated for their ease of scoring and preferential use in the forensic community [25,26]. Additionally, the observed allele size range (103-364 bp) for these markers allows for rapid data collection and accurate scoring due to these smaller fragment sizes [26]. The present system detected 63 alleles. The method of detection may be applied to discover more alleles in other plant samples, including fiber varieties.

The following definitions are used herein:

“Polymerase chain reaction” or “PCR” is a technique in which cycles of denaturation, annealing with primer, and extension with DNA polymerase are used to amplify the number of copies of a target DNA sequence by approximately 106 times or more. The polymerase chain reaction process for amplifying nucleic acid is disclosed in U.S. Pat. Nos. 4,683,195 and 4,683,202, which are incorporated herein by reference.

“Primer” is a single-stranded oligonucleotide or DNA fragment which hybridizes with a DNA strand of a locus in such a manner that the 3′ terminus of the primer may act as a site of polymerization using a DNA polymerase enzyme.

“Primer pair” is two primers including, primer 1 that hybridizes to a single strand at one end of the DNA sequence to be amplified and primer 2 that hybridizes with the other end on the complementary strand of the DNA sequence to be amplified.

“Primer site” the area of the target DNA to which a primer hybridizes.

“Multiplexing” is a capability to perform simultaneous, multiple determinations in a single assay process and a process to implement such a capability in a process is a “multiplexed assay.” Systems containing several loci are called multiplex systems described, for example, in U.S. Pat. No. 6,479,235 to Schumm, et al., U.S. Pat. No. 6,270,973 to Lewis, et al. and U.S. Pat. No. 6,449,562 to Chandler, et al.

“Cocktail” is a mixture of primer pairs selected to amplify one or more STR loci in a multiplex system.

“Isolated nucleic acid” is a nucleic acid which may or may not be identical to that of a naturally occurring nucleic acid. When “isolated nucleic acid” is used to describe a primer, the nucleic acid is not identical to the structure of a naturally occurring nucleic acid spanning at least the length of a gene. The primers herein have been designed to bind to sequences flanking STR loci in Cannabis sativa species. It is to be understood that primer sequences containing insertions or deletions in these disclosed sequences that do not impair the binding of the primers to these flanking sequences are also intended to be incorporated into the present invention.

Forensic Utility of STR Markers

Databases compiled by the present system will be used for drug trafficking and intelligence purposes and to track distribution patterns and growing operations. Additionally, databases are going to be necessary for gaining court acceptance of Cannabis DNA fingerprinting systems [12,28].

Recently, the forensic community has expressed considerable interest in non-human DNA fingerprinting methods for assisting in criminal investigations [27,28]. With the present STR system, forensic investigators will be able to generate genetic profiles of individual C. sativa plants and compare them to databases [12,28] or to suspected clonally propagated plants to determine if the profiles match. The identification of clonal growing operations and tracking distribution patterns of individual Cannabis plants has the greatest immediate potential for this system. The ability to generate matching genotypic profiles from plants confiscated from independent locations within the same residential area would support the hypothesis that the plants were coming from the same clonal growing operation.

Development of STR Markers

Of the seven arbitrary repeat motifs that were screened in this protocol, only three (AGC, AAAG, CCT) yielded sequences with sufficient flanking regions for primer development. Over two hundred individual positive clones were sequenced to find a total of 33 sequences that contained repeat motifs with at least five repeating units and sufficient flanking sequence on either side of the repeat. Of the 15 markers that were identified as polymorphic, only eight amplified consistently and were easy to score, with minimal stutter problems (Table 2).

| Locus Name | Repeat | Aplicon Size | Number of | |
|---|---|---|---|---|
| Dye Label^{a} | Motifs* | Range (bp) | Alleles | Multiplex Mix # |
| AAAG1 | (AAAG)6 | 103-135 | 16 | 1 |
| HEX | | | | |
| ACT1 | (ACT)6 | 218-224 | 3 | 1 |
| FAM | | | | |
| AGC8 | (AGC)5 | 264-279 | 6 | 1 |
| NED & FAM | | | | |
| AGC9 | (AGC)9 | 317-335 | 7 | 1 |
| HEX | | | | |
| AGC1 | (AGC)10 | 128-164 | 10 | 2 |
| FAM | | | | |
| AAAG5 | (AAAG)5 | 188-200 | 4 | 2 |
| NED | | | | |
| AAAG7 | (AAAG)6 | 242-266 | 7 | 3 |
| FAM | | | | |
| AAAG10 | (AAAG)5 | 352-364 | 4 | 3 |
| FAM | | | | |
| AGC6 | (AGC)6 | 200 & 221 | 2 | 3 |
| HEX | | | | |
| AGC10 | (AGC)43 | 273-327 | 15 | 3 |
| NED | | | | |

These primer sequences have herein been assigned SEQ ID NO: as follows:

| SEQ ID NO | Marker Name | |
|---|---|---|
| SEQ ID NO: 1 | AAAG1 | Forward primer |
| SEQ ID NO: 2 | AAAG1 | Reverse primer |
| SEQ ID NO: 3 | AAAG5 | Forward primer |
| SEQ ID NO: 4 | AAAG5 | Reverse primer |
| SEQ ID NO: 5 | AAAG6 | Forward primer |
| SEQ ID NO: 6 | AAAG6 | Reverse primer |
| SEQ ID NO: 7 | AAAG7 | Forward primer |
| SEQ ID NO: 8 | AAAG7 | Reverse primer |
| SEQ ID NO: 9 | AAAG10 | Forward primer |
| SEQ ID NO: 10 | AAAG10 | Reverse primer |
| SEQ ID NO: 11 | AAAG11 | Forward primer |
| SEQ ID NO: 12 | AAAG11 | Reverse primer |
| SEQ ID NO: 13 | AGC1 | Forward primer |
| SEQ ID NO: 14 | AGC1 | Reverse primer |
| SEQ ID NO: 15 | AGC3 | Forward primer |
| SEQ ID NO: 16 | AGC3 | Reverse primer |
| SEQ ID NO: 17 | AGC6 | Forward primer |
| SEQ ID NO: 18 | AGC6 | Reverse primer |
| SEQ ID NO: 19 | AGC8 | Forward primer |
| SEQ ID NO: 20 | AGC8 | Reverse primer |
| SEQ ID NO: 21 | AGC9 | Reverse primer |
| SEQ ID NO: 22 | AGC9 | Reverse primer |
| SEQ ID NO: 23 | AGC10 | Forward primer |
| SEQ ID NO: 24 | AGC10 | Reverse primer |
| SEQ ID NO: 25 | ACT1 | Forward primer |
| SEQ ID NO: 26 | ACT1 | Reverse primer |
| SEQ ID NO: 27 | CCT2 | Forward primer |
| SEQ ID NO: 28 | CCT2 | Reverse primer |

The polynucleotides of the present invention may be prepared by two general methods: (1) they may be synthesized from appropriate nucleotide triphosphates, or (2) they may be isolated from biological sources. Both methods utilize protocols well known in the art. The availability of nucleotide sequence information enables preparation of an isolated nucleic acid molecule of the invention by oligonucleotide synthesis. Synthetic oligonucleotides may be prepared by the phosphoramidite method employed in the Applied Biosystems 38A DNA Synthesizer or similar devices. The resultant construct may be purified according to methods known in the art, such as high performance liquid chromatography (HPLC). Complementary segments thus produced may be annealed such that each segment possesses appropriate cohesive termini for attachment of an adjacent segment. Adjacent segments may be ligated by annealing cohesive termini in the presence of DNA ligase to construct an entire long double-stranded molecule. A synthetic DNA molecule so constructed may then be cloned and amplified in an appropriate vector.

Total Genetic Diversity

A total of 295 C. sativa samples were analyzed and these samples included representatives from 33 countries or regions around the world. The greatest number of representative samples (188) came from the United States (Table 1). Virtually all of the samples in this study came either from drug confiscations or from known drug varieties of marijuana. Additionally, there were a small number of samples (<10) that were from known hemp or fiber varieties of Cannabis. DNA extracted from four dried samples that came from drug confiscations conducted in 1992 were included in the analyses. Although the DNA was fairly degraded, complete genotypic profiles were obtained for each of these four samples.

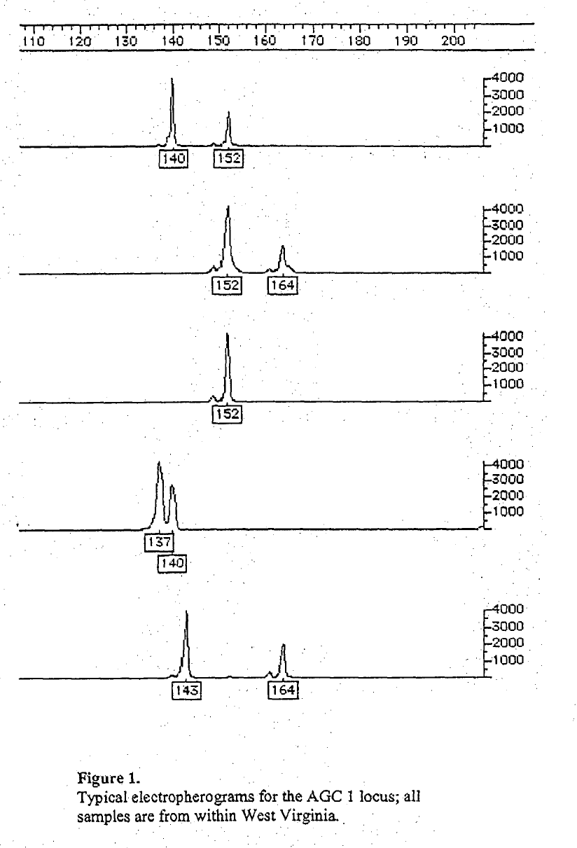

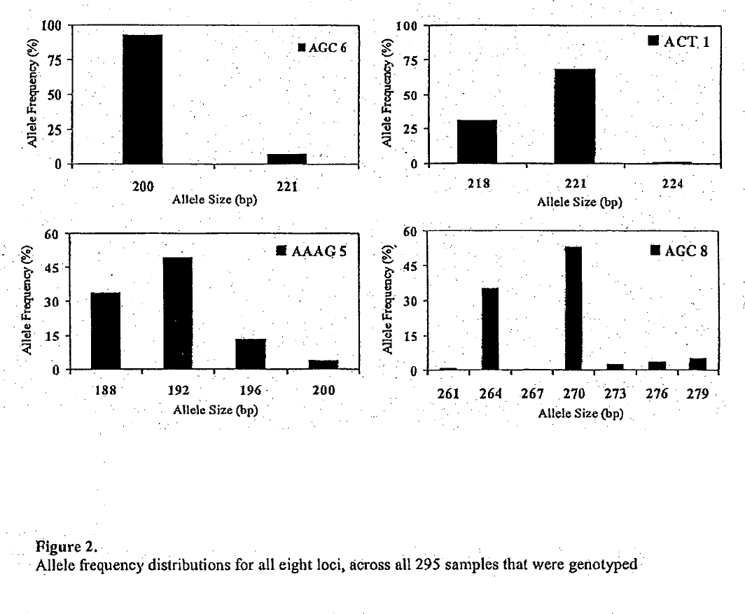

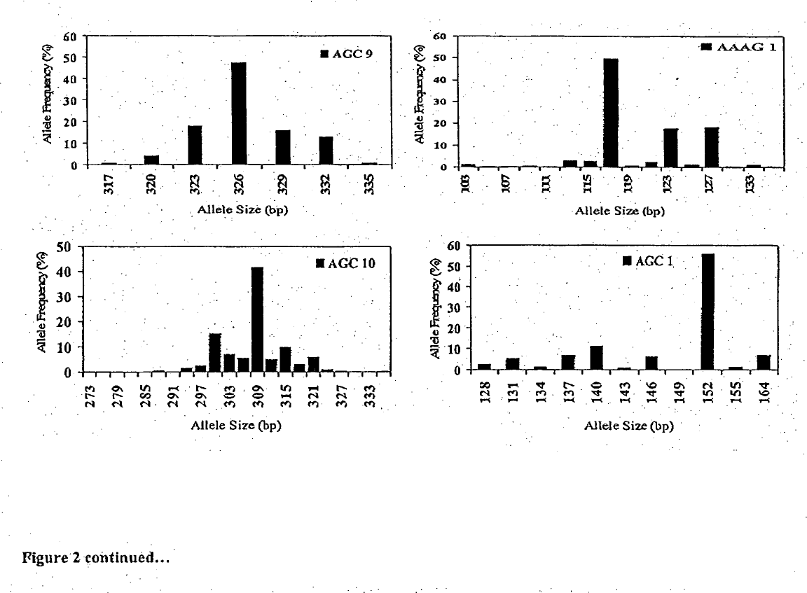

268 unique genotypes were found from the 295 C. sativa samples. For the samples that had at least one matching genotype from a different sample, it was noted that matches corresponded to samples with close geographic locations. All loci amplified robustly using 10 to 15 ng DNA and exhibited Mendelian inheritance, with a maximum of two alleles per locus. A total of 63 alleles were detected in this data set, with the number of alleles-per locus ranging from two at the AGC6 locus to 16 alleles at the AAAG1 locus (Table 2, FIG. 2). The overall observed heterozygosity (averaged across loci) was 0.41±±0.01 (mean±S.E.) while the expected heterozygosity was calculated to be 0.58±0.05, when averaged across all eight loci. The average heterozygosity per locus ranged from 0.21 to 0.79.

Allele Frequencies Per Locus

FIG. 2 shows the allele frequencies for each locus in this data set. All observed alleles within each locus, with the exception of two loci, varied by the addition or deletion of single repeat motifs, which is consistent with the assumption that STR loci mutate by insertions and deletions of repeat units. Exceptions of this assumption were observed at the AAAG1 and AGC6 loci. The AAAG1 locus was isolated from a sequence that appeared to contain a 4 bp repeat motif however; samples subjected to the fragment analyses appeared to vary by 2 bp instead of four. The AGC6 locus only had two observable allele sizes, spanning 21 bp, which would suggest a mutational event of seven repeat motif units.

The most diverse marker in this study was the AAAG1 locus, containing 16 alleles and spanning a 32 bp region of the genome, and all expected alleles were observed within this size range (FIG. 2). The second most diverse marker, AGC10 proved to be a noteworthy locus because of its large size range. At this locus we observed 15 alleles and an allelic size range from 273 bp to 336 (Table 2). All but seven of the 22 expected alleles were observed within this 63 bp size range.

Geographic Patterns

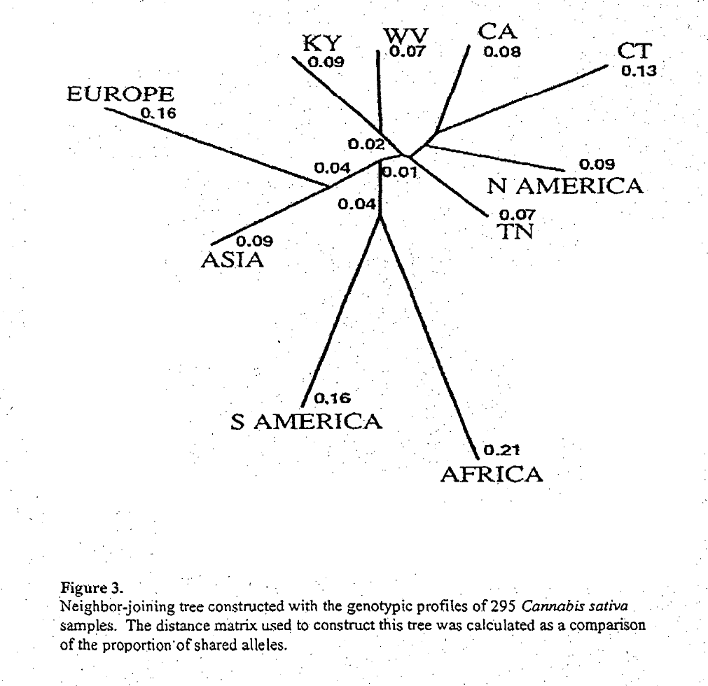

A neighbors-joining tree based on the proportion of shared alleles between samples was constructed. An assignment test was conducted to explore the potential utility of these markers for making geographic assignments based on a particular genotype. The results suggest a possible utility of these markers in detecting geographic differences on large, regional scales such as continents. The results of the neighbor-joining tree (FIG. 3) depict large-scale geographic clustering based on similar genotypes. All states within North America clustered together. Additionally, samples from Europe and Asia clustered together, while samples from South America and Africa clustered together.

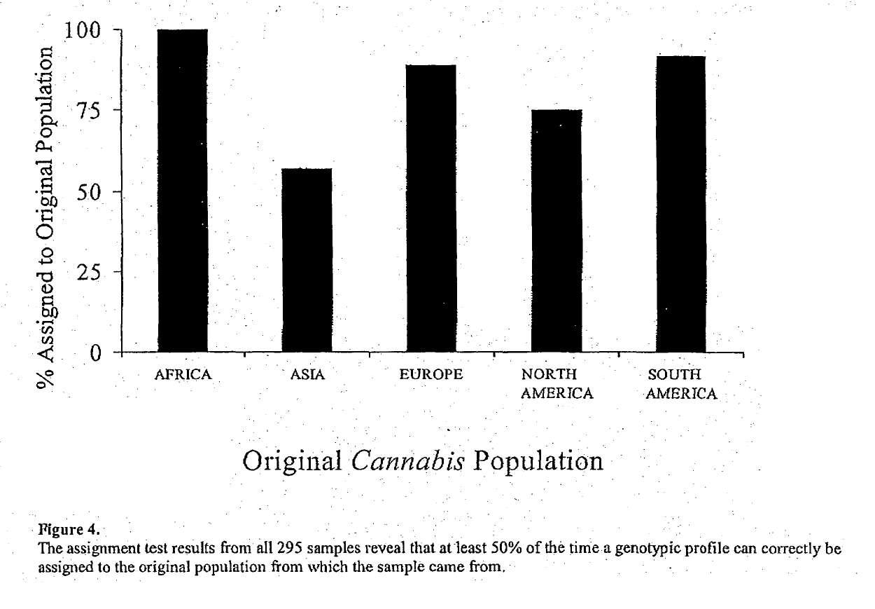

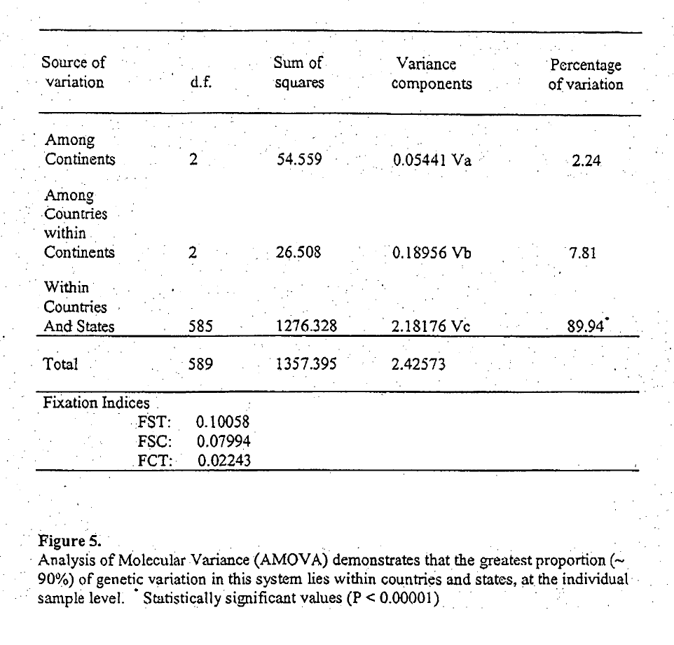

The results of the assignment test (FIG. 4) indicate that in general, genotypes can be correctly assigned to the right continent at least 50% of the time. Genotypes from the African population (13 samples) were correctly assigned to Africa in all instances; whereas genotypes from the Asian population (46 samples) were only correctly assigned to Asia 61% of the time (Table 1, FIG. 4). The North American population had the largest sample size (196 samples) and their genotypes were correctly assigned 72% of the time. This North American population, with its relatively large sample size, suggests that correct assignments to populations may increase with increasing sample size.

Genetic Diversity Among Individual Samples

We conducted an analysis of molecular variance (AMOVA) to determine the distribution of the genetic variation. Our findings revealed that the greatest proportion of genetic variation (˜90%) was among individual samples, within counties and states (Table 3). While the AMOVA did indicate that there were significant differences (P<0.0001) within countries and continents, this variation only accounted for approximately 8% of the total variance. This analysis also shows that the variation among the continents was not statistically significant at 2% (Table 3). The results of the AMOVA (Table 3) suggest that these markers are able to detect genetic differences between individual samples. Additionally, the number of unique genotypes observed, 268 out of 295 samples, also indicates that this system is capable of detecting a sizeable portion of the variation in the samples analyzed.

### EXPERIMENTAL DETAILS

DNA Extraction and Sample Preparation

Cannabis sativa DNA was ,extracted from dried leaf and flower material, in crime laboratories independent of our laboratory, by criminalistics professionals licensed to legally handle these plant samples. Virtually all of the samples came from drug confiscations or from known drug varieties of marijuana. Four different crime laboratories provided DNA samples for this study and there were two main extraction protocols that these agencies used. From these laboratories, we obtained a total of 295 samples with a wide geographic distribution, including representative samples from five different continents (see Table 1). For samples within the United States, the sample location generally refers to the location of the drug confiscation and cultivation. However, the international sample locations do not necessarily correspond to the location of cultivation. Rather these locations correspond to region where the seeds were obtained.

The majority of samples (240 samples) were extracted by the Appalachian H.I.D.T.A. Marijuana Signature Laboratory, Frankfort, Ky., using a modified CTAB (cetyltrimethylammonium bromide) protocol described by Weising et al. [15]. The remaining 55 samples were extracted in three independent laboratories, all using QIAGEN®'s DNeasy® plant mini kit (QIAGEN, Inc., Valencia, Calif., USA), following manufacturers recommendations for dried plant material. DNA samples were received in 100-150 μl of TE buffer [10 mM tris-HCl at pH 8.0, 1 mM EDTA (ethylenediaminetetraacetic acid)] and stored at −20° C. The approximate yield of each sample was assessed on a 0.7% agarose gel, where samples were compared to a Lambda Hind III DNA mass ladder of known concentrations (Invitrogen, Carlsbad, Calif., USA). All DNA samples were then diluted to approximately 10 to 15 ng/ul for the subsequent analyses.

Development of STR Markers

The STR (microsatellite) markers were developed using a modified magnetic bead protocol that was first described by Li et al. [16] and modified by Pearson [17]. Genomic DNA was digested from three different marijuana plants using an MboI restriction enzyme (Invitrogen; Carlsbad, Calif.). Sau 3a I Linkers A and B (SAULA: 5′-GCG GTA CCC GGG AAG CTT GG 3′ and SAULB: 5′ GAT CCC AAG CTT CCC GGG TAC CGC 3′) were ligated onto the digested genomic DNA and SAULA was used as a primer for subsequent polymerase chain reactions (PCR) [16]. The digested genomic DNA was amplified in multiple PCR reactions and concentrated to gain enough DNA for the following bead hybridization process.

Seven arbitrary repeat motifs were chosen as probes for the bead hybridization reactions based on a review by Cardle et al. [18] where they suggested that plants contain more AT-rich repeats than GC-rich repeats. The short tandem repeat (STR) probes were ordered from Integrated DNA Technologies (Coralville, Iowa, USA) with a biotin label on the 5′ end of the probes [(AGC)₈, (AAAG)₅, (CCT)₈, (AATT)₅, (ATT)₈, (GATA)₅, (ATGC)₅]. These repeat probes were, then added to a bead hybridization reaction to select for fragments of DNA that contain the repeat motif of the probe. The goal of this bead hybridization process was to allow the fragments containing repeats to anneal to the biotin-labeled probes. After the hybridization, the selected fragments were isolated from the rest of the genomic DNA using streptavidin coated magnetic beads, which bind to the biotin labeled probes. These fragments were then eluted and re-amplified using the SAULA primer in additional PCR reactions. The bead hybridization and PCR re-amplification processes were then repeated two additional times to enrich for genomic DNA containing the selected repeats.

Once the bead hybridization and selection process was completed, the repeat enriched DNA was then ligated into a pGEM-T vector from ProMega (Madison, Wis., USA) in order to begin the sequencing phase of this protocol. The vectors were cloned into electrocompetent E. coli cells that were then plated onto selective media containing [0.1 mg/mL ampicillin, 0.05 mg/mL X-Gal, and 1 mM IPTG] and positive clones were sequenced on an ABI PRISM® 377 DNA Sequencer (Applied Biosystems; Foster City, Calif., USA). The sequencing reactions were standard 20 μl reactions using the ABI PRISM® BigDye“ ” Terminators sequencing kits (Applied Biosystems; Foster City, Calif., USA) and 3.2 pmol of PCR product for template. Sequences containing repeat motifs and sufficient flanking sequence were used to design primers with PrimerSelect software (DNASTAR Inc.; Madison, Wis., USA).

Thirty-three primer pairs were screened on 3% agarose gels against 24 samples from different locations to identify polymorphic markers. Of the 33 markers that were initially screened, fifteen were determined to be polymorphic and we obtained these 15 markers with fluorescent dye labels. The fluorescent markers were tested on an ABI PRISM® 377 DNA Sequencer (Applied Biosystems; Foster City, Calif., USA) and seven of the 15 markers were eliminated due to problems with scoring or very low levels of polymorphism. The remaining eight markers (see Table 2) were tested in three multiplex reactions with two to four markers per mix and gels were run using GeneScan 2.1.1 (Applied Biosystems; Foster City, Calif., USA) collection software on an ABI PRISMS 377 DNA Sequencer (Applied Biosystems; Foster City, Calif., USA) Once multiplex reactions were optimized, 295 samples from individual plants were screened across all eight markers.

PCR Amplification and Fragment Analysis

The eight STR markers were optimized to amplify DNA in three 10 μl multiplex reactions (see Table 2). The multiplex mixes each contained approximately 10-15 ng of template from C. sativa in a 10 μl PCR including the following (final concentrations): 1×PCR buffer (Invitrogen; Carlsbad, Calif., USA), 3 mM MgCl₂ (Invitrogen, Carlsbad, Calif., USA), 200 μM dNTPs, 0.2 μM fluorescent forward primers, 0.2 μM unlabeled forward primers, 0.4 μM unlabeled reverse primers, and 1 unit Platinum DNA Taq Polymerase (Invitrogen; Carlsbad, Calif., USA). Amplification reactions were then carried out in 96-well microplates in a DNA engine thermocycler (MJ Research, Inc.; Waltham, Mass., USA) and the reaction contained a total of 35 cycles. The thermocycling conditions were as follows: an initial incubation of 95° C. for 5 min, next a cycle of denaturing at 95° C. for 3.0 sec, annealing at (59° C., 60° C., or 62° C.) for 30 sec, and extending at 72° C. for 30 sec, repeated for a total of 35 cycles, with a final extension of 72° C. for 2 min, and ending with a holding temperature of 15° C.

The PCR products were then diluted 1:10 with E-pure® purified water in preparation for fragment analysis on the ABI PRISM® 377 DNA Sequencer (Applied Biosystems; Foster City, Calif., USA). A size standard ladder mix was prepared with 0.75 μl deionized formamide, 0.25 μl of ROX labeled MapMarkers™ 1000 (BioVentures, Inc.; Murfreesboro, Tenn., USA), and 0.1 μl of blue dextran loading dye (supplied with the ROX size ladder). Approximately 1 μl of the size standard ladder mix was added to 1 μl of the diluted amplification products and denatured at 95° C. for 2 minutes. From this mixture, roughly 1.6 μl was loaded on a porous membrane comb (The Gel Company; San Francisco, Calif., USA) and then electrophoresed in a 5% polyacrylamide gel on'the ABI PRISM® 377 DNA Sequencer (Applied Biosystems; Foster City, Calif., USA) for 3.5 hours.

Scoring of STR Loci and Data Analysis

Electrophoresis data was collected automatically with GeneScan™ 2.1.1 software (PE Applied Biosystems; Foster City, Calif., USA); following collection, this software was also used to determine the allele sizes by implementing the local Southern method.

After initial scoring was completed, Genotyper™ software (Applied Biosystems; Foster City, Calif., USA) was used to confirm the allele scores. Banding patterns of homozygous and heterozygous genotypes were consistent with that of a single peak for homozygotes and double peaks for heterozygotes. Once all of the data scoring was complete, random samples were re-amplified and independently re-run to assess reproducibility and confirm the scoring and banding patterns.

Statistical analyses of the data were performed using a multitude of different analysis packages. An Excel add-in called The Excel Microsatellite Toolkit V3.1 [19] was used to calculate the number of matching genotypes, number of alleles, allele frequencies, and observed and expected heterozygosity. A distance matrix was generated in MICROSAT [20] based on the proportion of shared alleles, which was then input into PHYLIP [21] to construct a phylogenetic tree using a neighbor-joining algorithm. Genetic differentiation among continents was calculated in Arlequin V2.0 [22] using an Analysis of Molecular Variance (AMOVA). Finally an assignment test was performed in GenAlEx V5 [23].

### EXAMPLES

The following examples illustrate locus sequences for all fifteen polymorphic loci isolated from Cannabis sativa. Forward and Reverse primers are underlined. Variable regions are in lower case. *Most probes have an additional G added to the 5′ end of the oligo to increase adenylation. All sequences are 5′→3′

### Example 1

This example illustrates the amplicons produced during the amplification of STR locus AAAG 1 with multiplex cocktails comprising primer pairs SEQ ID NO: 1 and SEQ ID NO:2.

Sequence for AAAG 1 locus:
GCGGTACCCGGGAAGCTTGGGATCTAAACTGAGAGGTGGGTTTTGGTCAGAA
ACCGAAGACCTTTAGACCCAATATGAAGGAGaagaagaagaagaagaagaagaagaaa
gaaagaaagaaagaaagaaagAAAACACAGCTAGCAAAAGAAGTAAAGACAGGCAG
CCATCATTAATGGCAGAGAGATAGAGTGAGAAAGAGATAGAAAGGAGGAG
AGAGAGAGAGATAGAGAGTACAAGAAAGAAAGAGCAAAGCCAAGCTTCCCG
GGTACCGC
AAAG1F: GTCAGAAAGC GAAGACCTTT AGA [23 bp]
AAAG1R: GTAAAGACAG GCAGCCATC [19 bp]
AAAG1F (rev. comp.): TCTAAAGGTC TTCGCTTTCT GAC [23 bp]
AAAG1R (rev. comp.): GATGGCTGCC TGTCTTTAC [19 bp]
AAAG1 array: AAGAAGAAGA AGAAGAAGAA GAAGAAAGAA AGAAAGAAAG
AAAGAAAG [48 bp]
AAAG1 motif: (AAG)8 + (AAAG)6
AAAG1 amplicon: [275 bp]
GCGGTACCCG GGAAGCTTGG GATCTAAACT GAGAGGTGGG
TTTTGGTCAG AAAGCGAAGA CCTTTAGACC CAATATGAAG
GAGAAGAAGA AGAAGAAGAA GAAGAAGAAA GAAAGAAAGA
AAGAAAGAAA GAAAACACAG CTAGCAAAAG AAGTAAAGAC
AGGCAGCCAT CATTAATGGC AGAGAGATAG AGTGAGAAAG
AGATAGAAAG GAGGAGAGAG AGAGAGATAG AGAGTACAAG
AAAGAAAGAG CAAAGCCAAG CTTCCCGGGT ACCGC
AAAG1 (reverse compliment): [275 bp]
GCGGTACCCG GGAAGCTTGG CTTTGCTCTT TCTTTCTTGT. ACTCTCTATC
TCTCTCTCTC TCCTCCTTTC TATCTCTTTC TCACTCTATC TCTCTGCCAT
TAATGATGGC TGCCTGTCTT TACTTCTTTT GCTAGCTGTG TTTTCTTTCT
TTCTTTCTTT CTTTCTTTCT TCTTCTTCTT CTTCTTCTTC TTCTCCTTCA
TATTGGGTCT AAAGGTCTTC GCTTTCTGAC CAAAACCCAC CTCTCAGTTT
AGATCCCAAG CTTCCCGGGT ACCGC

### Example 2

This example illustrates the amplicons produced during the amplification of STR locus AAAG 5 with multiplex cocktails comprising primer pairs SEQ ID NO: 3 and SEQ ID NO:4.

Sequence for AAAG 5 locus:
GCGGTACCCGGGAAGCTTGGCATCAACTTGTCAAGCATTTAATATAAGATTG
GAATATATGTAACATCTCAATTAATGCTTATAGCCCATATGTTTTCTACTA
CTTCTTCTTTTTCAGTTGGTGTTATATAGCTTGATGATTACTTTCACGGTGTaaa
caaaagaagaagaaagaaagaaagaaagaaagaagACATGGGTTGAGCTGCTTCTGTATATG
TTGTTCCATGGAAGAACAAGAAGAAACAAAGTATTCCTGAAGTTGTGATAT
TTGTACCTTCATTGAAAATACCATTACAATCTGATCCCAAGCTTCCCGGGTAC
CGC
AAAG5F: TCAATTAATG CTTATAGCCC ATATGTTTTC TACTAC [36 bp]
AAAG5R: AGAACAAGAA GAAACAAAGT ATTCCTGAAG TTG [33 bp]
AAAG5F (rev. comp.): GTAGTAGAAA ACATATGGGC TATAAGCATT
AATTGA [36 bp]
AAAG5R (rev. comp.): CAACTTCAGG AATACTTTGT TTCTTCTTGT TCT
[33 bp]
AAAG5 array: AAACAAAAGA AGAAGAAAGA AAGAAAGAAA GAAAGAAG
[48 bp]
AAAG5 motif: (AAAC)1 + (AAAAG)1 + (AAG)2 + (AAAG)5 + (AAG)1
AAAG5 amplicon: [327 bp]
GCGGTACCCG GGAAGCTTGG CATCAACTTG TCAAGCATTT
AATATAAGAT TGGAATATAT GTAACATCTC AATTAATGCT TATAGCCCAT
ATGTTTTCTA CTACTTCTTC TTTTTCAGTT GGTGTTATAT AGCTTGATGA
TTACTTTCAC GGTGTAAACA AAAGAAGAAG AAAGAAAGAA
AGAAAGAAAG AAGACATGGG TTGAGCTGCT TCTGTATATG
TTGTTCCATG GAAGAACAAG AAGAAACAAA GTATTCCTGA
AGTTGTGATA TTTGTACCTT CATTGAAAAT ACCATTACAA TCTGATCCCA
AGCTTCCCGG GTACCGC
AAAG5 reverse compliment: [327 bp]
GCGGTACCCG GGAAGCTTGG GATCAGATTG TAATGGTATT
TTCAATGAAG GTACAAATAT CACAACTTCA GGAATACTTT GTTTCTTCTT
GTTCTTCCAT GGAACAACAT ATACAGAAGC AGCTCAACCC ATGTCTTCTT
TCTTTCTTTC TTTCTTTCTT CTTCTTTTGT TTACACCGTG AAAGTAATCA
TCAAGCTATA TAACACCAAC TGAAAAAGAA GAAGTAGTAG
AAAACATATG GGCTATAAGC ATTAATTGAG ATGTTACATA TATTCCAATC
TTATATTAAA TGCTTGACAA GTTGATGCCA AGCTTCCCGG GTACCGC

### Example 3

This example illustrates the amplicons produced during the amplification of STR locus AAAG 6 with multiplex cocktails comprising primer pairs SEQ ID NO: 5 and SEQ ID NO: 6.

Sequence for AAAG 6 locus:
GCGGTACCCGGGAAGCTTGGCTTAGATTAAGAATATTTGTAGTTTCGTACTTG
TATTCCTTGCCTTTTTCAAGATTTCTT
GCTTGTTTAGGGTATCTGCCATTTTTCTTTCTCCTTTCAGAGCTTCTTCTAATC
CAAGATTCCCAAGATGAGCAATTGTC
TTTTCACCCCACAGACTGAAATTGTTTTTGCCATTGATTTCCTCCTCCTCAT
ACTTCTCCAAAGACATTATTGAACAAATAAGaaagaaagaaagaaagaaagaaagaaaga
aagaaagAAAAACTTATGGCCAGTAAGCGTTTCCCTTGTTGGTTACCTTTCTTCA
GTCTTTGAGGAATTCATTCGAACACTCTGTCAACCTCAACTGGTTTCTTCAAA
CTCTAATCTGAAACCTGGCTCTTGATACCAGTTTGTGAGGATTGGTCTCCTCT
TCTCCAATCTCAGATCCCAAGCTTCCCGGGTACCGC
AAAG6F: TTTGCCATTG ATTTCCTCCT CCTCATAC [28 bp]
AAAG6R: AGATCCCAAG CTTCCCGGGT ACC [23 bp]
AAAG6F (rev. comp.): GTATGAGGAG GAGGAAATCA ATGGCAAA [28 bp]
AAAG6R (rev. comp.): GGTACCCGGG AAGCTTGGGA TCT [23 bp]
AAAG6 array: AAAGAAAGAA AGAAAGAAAG AAAGAAAGAA AGAAAG
[36 bp]
AAAG6 motif: (AAAG)9
AAAG6 locus: [469 bp]
GCGGTACCCG GGAAGCTTGG CTTAGATTAA GAATATTTGT AGTTTCGTAC
TTGTATTCCT TGCCTTTTTC AAGATTTCTT GCTTGTTTAG GGTATCTGCC
ATTTTTCTTT CTCCTTTCAG AGCTTCTTCT AATCCAAGAT TCCCAAGATG
AGCAATTGTC TTTTCACCCC ACAGACTGAA ATTGTTTTTG CCATTGATTT
CCTCCTCCTC ATACTTCTCC AAAGACATTA TTGAACAAAT
AAGAAAGAAA GAAAGAAAGA AAGAAAGAAA GAAAGAAAGA
AAAACTTATG GCCAGTAAGC GTTTCCCTTG TTGGTTACCT TTCTTCAGTC
TTTGAGGAAT TCATTCGAAC ACTCTGTCAA CCTCAACTGG TTTCTTCAAA
CTCTAATCTG AAACCTGGCT CTTGATACCA GTTTGTGAGG ATTGGTCTCC
TCTTCTCCAA TCTCAGATCC CAAGCTTCCC GGGTACCGC
AAAG6 reverse compliment: [469 bp]
GCGGTACCCG GGAAGCTTGG GATCTGAGAT TGGAGAAGAG
GAGACCAATC CTCACAAACT GGTATCAAGA GCCAGGTTTC
AGATTAGAGT TTGAAGAAAC CAGTTGAGGT TGACAGAGTG
TTCGAATGAA TTCCTCAAAG ACTGAAGAAA GGTAACCAAC
AAGGGAAACG CTTACTGGCC ATAAGTTTTT CTTTCTTTCT TTCTTTCTTT
CTTTCTTTCT TTCTTTCTTA TTTGTTCAAT AATGTCTTTG GAGAAGTATG
AGGAGGAGGA AATCAATGGC AAAAACAATT TCAGTCTGTG
GGGTGAAAAG ACAATTGCTC ATCTTGGGAA TCTTGGATTA
GAAGAAGCTC TGAAAGGAGA AAGAAAAATG GCAGATACCC
TAAACAAGCA AGAAATCTTG AAAAAGGCAA GGAATACAAG
TACGAAACTA CAAATATTCT TAATCTAAGC CAAGCTTCCC GGGTACCGC

### Example 4

This example illustrates the amplicons produced during the amplification of STR locus AAAG 7 with multiplex cocktails comprising primer pairs SEQ ID NO: 7 and SEQ ID NO: 8.

Sequence for AAAG 7 locus:
GCGGTACCCGGGAAGCTTGGATCAGAAAGACAAGACAAGATAGGGACTACT
ACAAAGATTCCCACACTCAATAATGCAAATACAATTATTAGTACTAATAAT
GAAAACAACATCAAATTAAAGAAAAACCATAGAAGaaaacaaaaagaaaagaaagaaa
gaaagaaagATAGATAGATACCTGGTAGTGGGTTGGTTGGTTGGTGGTGATGAGT
ACTGAAATGGAAGACAATGAAAGGAGAAGGGGTTTACAGTGTTAACACTAT
AGTAAGGATTTGGTTTTCGGCTTTCGTTCTTTTAAGGAAGATGGGTGTTTG
AGAATGGATTGAGTAGTACAAGTCCAAATTCACAAGCAATTGCAGAGGCAGA
CGATGACTTCTTCAAATTCATAAGCAAGTGCCGAGGCAACCGATCCCAAGCT
TCCCGGGTACCGC
AAAG7F: CTACAAAGAT TCCCACACTC AATAATGCAA ATACAA [36 bp]
AAAG7R: AGTAAGGATT TGGTTTTCGG CTTTCGTTCT T [31 bp]
AAAG7F (rev. comp.): TTGTATTTGC ATTATTGAGT GTGGGAATCT TTGTAG
[36 bp]
AAAG7R (rev. comp.): AAGAACGAAA GCCGAAAACC AAATCCTTAC T [31 bp]
AAAG7 array: AAAACAAAAA GAAAAGAAAG AAAGAAAGAA AG [32 bp]
AAAG7 motif: (AAAAAG)1 + (AAAAG)1 + (AAAG)4
AAAG7 locus: [434 bp]
GCGGTACCCG GGAAGCTTGG ATCAGAAAGA CAAGACAAGA
TAGGGACTAC TACAAAGATT CCCACACTCA ATAATGCAAA
TACAATTATT AGTACTAATA ATGAAAACAA CATCAAATTA
AAGAAAAACC ATAGAAGAAA ACAAAAAGAA AAGAAAGAAA
GAAAGAAAGA TAGATAGATA CCTGGTAGTG GGTTGGTTGG
TTGGTGGTGA TGAGTACTGA AATGGAAGAC AATGAAAGGA
GAAGGGGTTT ACAGTGTTAA CACTATAGTA AGGATTTGGT TTTCGGCTTT
CGTTCTTTTA AGGAAGATGG GTGTTTGAGA ATGGATTGAG
TAGTACAAGT CCAAATTCAC AAGCAATTGC AGAGGCAGAC
GATGACTTCT TCAAATTCAT AAGCAAGTGC
CGAGGCAACC GATCCCAAGC TTCCCGGGTA CCGC
AAAG7 reverse compliment: [434 bp]
GCGGTACCCG GGAAGCTTGG GATCGGTTGC CTCGGCACTT
GCTTATGAAT TTGAAGAAGT CATCGTCTGC CTCTGCAATT GCTTGTGAAT
TTGGACTTGT ACTACTCAAT CdATTCTCAA ACACCCATCT TCCTTAAAAG
AACGAAAGCC GAAAACCAAA TCCTTACTAT AGTGTTAACA
CTGTAAACCC CTTCTCCTTT CATTGTCTTC CATTTCAGTA CTCATCACCA
CCAACCAACC AACCCACTAC CAGGTATCTA TCTATCTTTC TTTCTTTCTT
TCTTTTCTTT TTGTTTTCTT CTATGGTTTT TCTTTAATTT GATGTTGTTT
TCATTATTAG TACTAATAAT TGTATTTGCA TTATTGAGTG TGGGAATCTT
TGTAGTAGTC CCTATCTTGT CTTGTCTTTC TGATCCAAGC TTCCCGGGTA
CCGC

### Example 5

This example illustrates the amplicons produced during the amplification of STR locus AAAG 10 with multiplex cocktails comprising primer pairs SEQ ID NO: 9 and SEQ ID NO: 10.

Sequence for AAAG 10 locus:
GCGGTACCCGGGAAGCTTGGATAACAAAAATTCATACATAAGGCACGAAG
AGATAGACATAGaaagaaagaaagaaagaaagGAAAAAAAAAAATACTAAAACGAC
ATACACGGTCTTAGAGGACGAAGCAACTGCGCCGCCGCCGGTGACTGGGTTC
CT
TGGTCGAGAGGGAAAAAGAGGTTTTTGGTCTCTCTGACTCTGTTGTGCAGTGA
GATGAGGAGTGGAGAGTCGGATAGCATCATTTTTACACTAACTGAGAAGAAC
AACTTTTGATTTGGTTTGGTTTAAGGAAGAAAAAATCCCACATCGACTTGTTA
TAGCTTTTTTAATATGTTTATATTGATTACTTTATACAGTCCTATCGCCGGG
TCCAAGCTTCCCGGGTACCGC
AAAG10F: CAAAAATTCA TACATAAGGC ACGAAGAGAT AGACA [35 bp]
AAAG10R: TTTATACAGT CCTATCGCCG GGTCCAA [27 bp]
AAAG10F (rev. comp.): TGTCTATCTC TTCGTGCCTT ATGTATGAAT TTTTG
[35 bp]
AAAG10R (rev. comp.): TTGGACCCGG CGATAGGACT GTATAAA [27 bp]
AAAG10 array: AAAGAAAGAA AGAAAGAAAG [20 bp]
AAAG10 motif: (AAAG)5
AAAG10 locus: [391 bp]
GCGGTACCCG GGAAGCTTGG ATAACAAAAA TTCATACATA
AGGCACGAAG AGATAGACAT AGAAAGAAAG AAAGAAAGAA
AGGAAAAAAA AAAATACTAA AACGACATAC ACGGTCTTAG
AGGACGAAGC AACTGCGCCG CCGCCGGTGA CTGGGTTCCT
TGGTCGAGAG GGAAAAAGAG GTTTTTGGTC TCTCTGACTC TGTTGTGCAG
TGAGATGAGG AGTGGAGAGT CGGATAGCAT CATTTTTACA
CTAACTGAGA AGAACAACTT TTGATTTGGT TTGGTTTAAG
GAAGAAAAAA TCCCACATCG ACTTGTTATA GCTTTTTTAA TATGTTTATA
TTGATTACTT TATACAGTCC TATCGCCGGG TCCAAGCTTC CCGGGTACCG
C
AAAG10 reverse compliment: [391 bp]
GCGGTACCCG GGAAGCTTGG ACCCGGCGAT AGGACTGTAT
AAAGTAATCA ATATAAACAT ATTAAAAAAG CTATAACAAG
TCGATGTGGG ATTTTTTCTT CCTTAAACCA AACCAAATCA AAAGTTGTTC
TTCTCAGTTA GTGTAAAAAT GATGCTATCC GACTCTCCAC TCCTCATCTC
ACTGCACAAC AGAGTCAGAG AGACCAAAAA CCTCTTTTTC
CCTCTCGACC AAGGAACCCA GTCACCGGCG GCGGCGCAGT
TGCTTCGTCC TCTAAGACCG TGTATGTCGT TTTAGTATTT TTTTTTTTCC
TTTCTTTCTT TCTTTCTTTC TATGTCTATC TCTTCGTGCC TTATGTATGA
ATTTTTGTTA TCCAAGCTTC CCGGGTACCG C

### Example 6

This example illustrates the amplicons produced during the amplification of STR locus AAAG 11 with multiplex cocktails comprising primer pairs SEQ ID NO: 11 and SEQ ID NO: 12.

Sequence for AAAG 11 locus:
TTGCGGTACCCGGGAAGCTTGGATCTTAAAAGTTCAGGGGGCAAAAATCATA
ATTAGCCTATTGTTAATAATAGACCCTCCTAAAAATCGTTTTGCAAAATAACA
TTCTTTTCATAATTGTTTGCAAAATAATCTTTCTCTAGAATCCAAATAGTAT
TGAGAATTTTTAACAAAGTATTTGGAATTCTTAACAAAATGTTAGATTGTGAA
GGTGCTAGAAAGGTCATTTTTTGTTAAAAATTATCATCTATCAATTACTCATG
ATAGATTGTTGGAATAGAATCACAAGTTTTTGTTACACTATTATGTGGAGTGA
TTGGTGAAAATACACTTATTATGCAAATTGTACATAAAAAGAAGGaaagaaagaa
agaaagTCTATTTCACCAAACAAAAGAAACACCTTTATTATGTGAAAGTGATTG
ATGCATAAAGACTAATAATGCAGGATTTGAAGAGCCTTTGAGAGCATGTTGT
GGTCATGGTGGGAAGTATAATTTTAATAAGAaCATTGGATGTGGGGGCAAG
AAAATGGTCCATGGGAAAGAGATTTTGGTGGGAAAGGCTTGTAAAGATCCAA
GCTTCCCGGGTACCGC
AAAG11F: TTTTCATAAT TGTTTGCAAA ATAATCTTTC TCTAGAA [37 bp]
AAAG11R: GTTGTGGTCA TGGTGGGAAG TATAATTTTA ATA [33 bp]
AAAG11F (rev. comp.): 3TCTAGAGAA AGATTATTTT GCAAACAATT
ATGAAAA [37 bp]
AAAG11R (rev. comp.): TATTAAAATT ATACTTCCCA CCATGACCAC AAC
[33 bp]
AAAG11 array: AAAGAAAGAA AGAAAG [16 bp]
AAAG11 motif: (AAAG)4
AAAG11 locus: [596 bp]
TTGCGGTACC CGGGAAGCTT GGATCTTAAA AGTTCAGGGG
GCAAAAATCA TAATTAGCCT ATTGTTAATA ATAGACCCTC CTAAAAATCG
TTTTGCAAAA TAACATTCTT TTCATAATTG TTTGCAAAAT AATCTTTCTC
TAGAATCCAA ATAGTATTGA GAATTTTTAA CAAAGTATTT GGAATTCTTA
ACAAAATGTT AGATTGTGAA GGTGCTAGAA AGGTCATTTT
TTGTTAAAAA TTATCATCTA TCAATTACTC ATGATAGATT GTTGGAATAG
AATCACAAGT TTTTGTTACA CTATTATGTG GAGTGATTGG TGAAAATACA
CTTATTATGC AAATTGTACA TAAAAAGAAG GAAAGAAAGA
AAGAAAGTCT ATTTCACCAA ACAAAAGAAA CACCTTTATT
ATGTGAAAGT GATTGATGCA TAAAGACTAA TAATGCAGGA
TTTGAAGAGC CTTTGAGAGC ATGTTGTGGT CATGGTGGGA AGTATAATTT
TAATAAGAAC ATTGGATGTG GGGGCAAGAA AATGGTCCAT
GGGAAAGAGA TTTTGGTGGG
AAAGGCTTGT AAAGATCCAA GCTTCCCGGG TACCGC
AAAG11 reverse compliment: [596 bp]
GCGGTACCCG GGAAGCTTGG ATCTTTACAA GCCTTTCCCA CCAAAATCTC
TTTCCCATGG ACCATTTTCT TGCCCCCACA TCCAATGTTC TTATTAAAAT
TATACTTCCC ACCATGACCA CAACATGCTC TCAAAGGCTC TTCAAATCCT
GCATTATTAG TCTTTATGCA TCAATCACTT TCACATAATA AAGGTGTTTC
TTTTGTTTGG TGAAATAGAC TTTCTTTCTT TCTTTCCTTC TTTTTATGTA
CAATTTGCAT AATAAGTGTA TTTTCACCAA TCACTCCACA TAATAGTGTA
ACAAAAACTT GTGATTCTAT TCCAACAATC TATCATGAGT AATTGATAGA
TGATAATTTT TAACAAAAAA TGACCTTTCT AGCACCTTCA CAATCTAACA
TTTTGTTAAG AATTCCAAAT ACTTTGTTAA AAATTCTCAA TACTATTTGG
ATTCTAGAGA AAGATTATTT TGCAAACAAT TATGAAAAGA ATGTTATTTT
GCAAAACGAT TTTTAGGAGG GTCTATTATT AACAATAGGC TAATTATGAT
TTTTGCCCCC TGAACTTTTA AGATCCAAGC TTCCCGGGTA CCGCAA

### Example 7

This example illustrates the amplicons produced during the amplification of STR ocus AGC 1 with multiplex cocktails comprising primer pairs SEQ ID NO: 13 and SEQ ID NO: 14.

Sequence for AGC 1 locus:
GGGCCCGACGTCGCATGCTCCCGGCCGCCATGGCCGCGGGATTTACCCGGGA
AGCTTGGATAAGACCATGGCAAGAAAAGATGAGCAACAGAATGTGGTAATT
CAATACAAACAGAACACAAGTCGAATGGATAATAATAATAAGAAGAAACAG
TTGCCAAGCTGTCAAAAGAAATCACAGAACAATTTAGAGTTACAACAACCAT
TCGTGCCTGGAAAATTAGTATCACAAGATAATGGAAAACAAGTTTTACAGAC
AAGAAAACAAAAGGGTAGCACTGGTAGTAGTGAAGTTATGGCAAAGAGTGT
ATCGAAACCTGTCCGTGATGGAACAAATTTTCAACAGAagcagcagcagcagcagca
gcagcagcagcCACAGTCTAACCAAGAAAAGTTGAATAAGAAAGGTTTGAAAAAA
GGTACTAATACAGACGATGTGGTGGGGGTAGAAAGAAATTTGGCTGAATC
CAATTTCGTTAAGGAATACAACAATCGAAGCCCGGATCCCAAGCTTCCCGGG
TACCGC
AGC1F: CAAAGAGTGT ATCGAAACCT GTC [23 bp]
AGC1R: GTACTAATAC AGACGATGTG GTGGG [25 bp]
AGC1F (rev. comp.): GACAGGTTTC GATACACTCT TTG [23 bp]
AGC1R (rev. comp.): CCCACCACAT CGTCTGTATT AGTAC [25 bp]
AGG1 array: AGCAGCAGCA GCAGCAGCAG CAGCAGCAGC [30 bp]
AGC1 motif: (AGC)10
AGC1 locus: [529 bp]
GGGCCCGACG TCGCATGCTC CCGGCCGCCA TGGCCGCGGG
ATTTACCCGG GAAGCTTGGA TAAGACCATG GCAAGAAAAG
ATGAGCAACA GAATGTGGTA ATTCAATACA AACAGAACAC
AAGTCGAATG GATAATAATA ATAAGAAGAA ACAGTTGCCA
AGCTGTCAAA AGAAATCACA GAACAATTTA GAGTTACAAC
AACCATTCGT GCCTGGAAAA TTAGTATCAC AAGATAATGG
AAAACAAGTT TTACAGACAA GAAAACAAAA GGGTAGCACT
GGTAGTAGTG AAGTTATGGC AAAGAGTGTA TCGAAACCTG
TCCGTGATGG AACAAATTTT CAACAGAAGC AGCAGCAGCA
GCAGCAGCAG CAGCAGCCAC AGTCTAACCA AGAAAAGTTG
AATAAGAAAG GTTTGAAAAA AGGTACTAAT ACAGACGATG
TGGTGGGGGT AGAAAGAAAT TTGGCTGAAT CCAATTTCGT
TAAGGAATAC AACAATCGAA GCCCGGATCC CAAGCTTCCC GGGTACCGC
AGC1 reverse compliment: [529 bp]
GCGGTACCCG GGAAGCTTGG GATCCGGGCT TCGATTGTTG TATTCCTTAA
CGAAATTGGA TTCAGCCAAA TTTCTTTCTA CCCCCACCAC ATCGTCTGTA
TTAGTACCTT TTTTCAAACC TTTCTTATTC AACTTTTCTT GGTTAGACTG
TGGCTGCTGC TGCTGCTGCT GCTGCTGCTG CTTCTGTTGA AAATTTGTTC
CATCACGGAC AGGTTTCGAT ACACTCTTTG CCATAACTTC ACTACTACCA
GTGCTACCCT TTTGTTTTCT TGTCTGTAAA ACTTGTTTTC CATTATCTTG
TGATACTAAT TTTCCAGGCA CGAATGGTTG TTGTAACTCT AAATTGTTCT
GTGATTTCTT TTGACAGCTT GGCAACTGTT TCTTCTTATT ATTATTATCC
ATTCGACTTG TGTTCTGTTT GTATTGAATT ACCACATTCT GTTGCTCATC
TTTTCTTGCC ATGGTCTTAT CCAAGCTTCC CGGGTAAATC CCGCGGCCAT
GGCGGCCGGG AGCATGCGAC GTCGGGCCC

### Example 8

This example illustrates the amplicons produced during the amplification of STR locus AGC 3 with multiplex cocktails comprising primer pairs SEQ ID NO: 15 and SEQ ID NO: 16.

Sequence for AGC 3 locus:
GCGGTACCCGGGAAGCTTGGATCCTGGTAAAATAAAATTCCAACAGTTCACA
AGTACCAAACACAACTCCCCCTGGAAAAGGGTCAAGATTTTGTCCAAACAAA
CAGTTAAAAATCAAAATATTACTCCCCCTTTTTGTTTATCTAAGGGCCAAAGA
TAACAAACATGAAAATATAGTAATATGTCCAACAAAAGCAAAGAAAGAAA
AAAAAACTTAGTCTCTGTAAAGCTTGACCAAGGTGGACAACTGCTTTGACAT
CTTTTGCTGAACTTCCTCCATGGCAGCAAGACGATTGTTCACCAGCTGAACCT
CATTCTTGACGTCATGGATTTCTGCGGAAGCAGAATTCGAGCTTGCAACagcag
cagcagcaccagcTTTAGGCCATTTTTGAAACACACCATCAAAGTATTTCGAGGGTT
GGAATGTAGGTCCAATGATAGGGGGCTCAAGTGTTTCATGTGATTGGGCCA
CATTCTTTTGGGAAGATAAAACCTTATAGATTAGATTTGGAAATACAAGTTTA
AAGGTTGGCTTTTTATCTCTTCGGAAAGAAACAATCTGGTTCAGAATGTGTGA
GGCCAAATCAATTGAAGCTCCAGAGGTGATGCGGTATAAGAATGATGCCACA
TCTTGAGACACTACGGTCTTGTTGGAGT
AGC3F: ATAGTAATAT GTCCAACAAA AGCAAAGAAA GAAAAA [36 bp]
AGC3R: CAAGTGTTTC ATGTGATTGG GCCAC [25 bp]
AGC3F (rev. comp.): TTTTTCTTTC TTTGCTTTTG TTGGACATAT TACTAT
[36 bp]
AGC3R (rev. comp.): GTGGCCCAAT CACATGAAAC ACTTG [25 bp]
AGC3 array: AGCAGCAGCA GCACCAGC [18 bp]
AGC3 locus: [660bp]
GCGGTACCCG GGAAGCTTGG ATCCTGGTAA AATAAAATTC
CAACAGTTCA CAAGTACCAA ACACAACTCC CCCTGGAAAA
GGGTCAAGAT TTTGTCCAAA CAAACAGTTA AAAATCAAAA
TATTACTCCC CCTTTTTGTT TATCTAAGGG CCAAAGATAA CAAACATGAA
AATATAGTAA TATGTCCAAC AAAAGCAAAG AAAGAAAAAA
AAACTTAGTC TCTGTAAAGC TTGACCAAGG TGGACAACTG CTTTGACATC
TTTTGCTGAA CTTCCTCCAT GGCAGCAAGA CGATTGTTCA CCAGCTGAAC
CTCATTCTTG ACGTCATGGA TTTCTGCGGA AGCAGAATTC GAGCTTGCAA
CAGCAGCAGC AGCACCAGCT TTAGGCCATT TTTGAAACAC
ACCATCAAAG TATTTCGAGG GTTGGAATGT AGGTCCAATG
ATAGGGGGCT CAAGTGTTTC ATGTGATTGG GCCACATTCT TTTGGGAAGA
TAAAACCTTA TAGATTAGAT TTGGAAATAC AAGTTTAAAG GTTGGCTTTT
TATCTCTTCG GAAAGAAACA ATCTGGTTCA GAATGTGTGA
GGCCAAATCA ATTGAAGCTC CAGAGGTGAT GCGGTATAAG
AATGATGCCA CATCTTGAGA CACTACGGTC TTGTTGGAGT
AGC3 reverse compliment: [660 bp]
ACTCCAACAA GACCGTAGTG TCTCAAGATG TGGCATCATT CTTATACCGC
ATCACCTCTG GAGCTTCAAT TGATTTGGCC TCACACATTC TGAACCAGAT
TGTTTCTTTC CGAAGAGATA AAAAGCCAAC CTTTAAACTT GTATTTCCAA
ATCTAATCTA TAAGGTTTTA TCTTCCCAAA AGAATGTGGC CCAATCACAT
GAAACACTTG AGCCCCCTAT CATTGGACCT ACATTCCAAC CCTCGAAATA
CTTTGATGGT GTGTTTCAAA AATGGCCTAA AGCTGGTGCT GCTGCTGCTG
TTGCAAGCTC GAATTCTGCT TCCGCAGAAA TCCATGACGT
CAAGAATGAG GTTCAGCTGG TGAACAATCG TCTTGCTGCC
ATGGAGGAAG TTCAGCAAAA GATGTCAAAG CAGTTGTCCA
CCTTGGTCAA GCTTTACAGA GACTAAGTTT TTTTTTCTTT CTTTGCTTTT
GTTGGACATA TTACTATATT TTCATGTTTG TTATCTTTGG CCCTTAGATA
AACAAAAAGG GGGAGTAATA TTTTGATTTT TAACTGTTTG TTTGGACAAA
ATCTTGACCC TTTTCCAGGG GGAGTTGTGT TTGGTACTTG TGAACTGTTG
GAATTTTATT TTACCAGGAT CCAAGCTTCC CGGGTACCGC

### Example 9

This example illustrates the amplicons produced during the amplification of STR locus AGC 6 with multiplex cocktails comprising primer pairs. SEQ ID NO: 17 and SEQ ID NO: 18.

Sequence for AGC 6 locus:
TAGWTGAGCCCGACGTCGCATGCTCCCGGCCGCCATGGCCCGCGGGATTGCG
GTACCCGGGAAGCTTGGCAATATACAATCTSAGKTCACTCTCTGCTTTCCCAA
GCAGCCCTTGTTTGCAAGTATGCTCAAGACCAACGAAGTACCAGCACTGAGG
CTTGAATGCATGAGTAAAATGTAAAGAAGCCTTCTTTCCCTTTCCGCTTCCAC
TTTCCACCACCAAAAACTGTGCATGGAAGTATGCCTCTATTCCCTGGTTGTCA
GCAGACAAGAAACTGAACAGACGTGGCATATGCGCTGTTCCTTCACCTGC
AAGCGCACTGGCAGCAGCAGCAGCCGACATAGCTGAAGATTTTCCTGACTTag
cagcagcagcagcagcTATTGCAGCAGCAGCAGTTGCTGTATTTAACGTATCAGCAA
ATGATTCAATGTAAATCCATGTTGCAAATGCATACCCATTAGTGAACGGCC
ATCGGCTTTCCCCTGGACCAAGCAAACCAGAGCTTTCACCATCAAACTCAAA
AGTACATGCTGGTCCCTTTGACTCCTTTCCACTAACTGCCTTCTCCAAAGCAA
TCATTAAGCGAGCTGACCAAACAGTGCTAAGTGTTCTTGTGATGACTTGAAA
CCATCTATGCAAATCGATGACACTAAGTG
AGC6F: AGACGTGGCA TATGCGCTGT TCCTTCA [27 bp]
AGC6R: GCATACCCAT TAGTGAACGG CCATCGGC [28 bp]
AGC6F (rev. comp.): TGAAGGAACA GCGCATATGC CACGTCT [27 bp]
AGC6R (rev. comp.): GCCGATGGCC GTTCACTAAT GGGTATGC [28 bp]
AGC6 array: AGGAGGAGGA GCAGCAGC [18 bp]
AGC6 motif: (AGC)6
AGC6 locus: [663 bp]
TACWTGAGCC CGACGTCGCA TGCTCCCGGC CGCCATGGCC
CGCGGGATTG CGGTACCCGG GAAGCTTGGC AATATACAAT
CTSAGKTCAC TCTCTGCTTT CCCAAGCAGC CCTTGTTTGC AAGTATGCTC
AAGACCAACG AAGTACCAGC ACTGAGGCTT GAATGCATGA
GTAAAATGTA AAGAAGCCTT CTTTCCCTTT CCGCTTCCAC TTTCCACCAC
CAAAAACTGT GCATGGAAGT ATGCCTCTAT TCCCTGGTTG TCAGCAGACA
AGAAACTGAA CAGACGTGGC ATATGCGCTG TTCCTTCACC
TGCAAGCGCA CTGGCAGCAG CAGCAGCCGA CATAGCTGAA
GATTTTCCTG ACTTAGCAGC AGCAGCAGCA GCTATTGCAG
CAGCAGCAGT TGCTGTATTT AACGTATCAG CAAATGATTC AATGTAAATC
CATGTTGCAA ATGCATACCC ATTAGTGAAC GGCCATCGGC TTTCCCCTGG
ACCAAGCAAA CCAGAGCTTT CACCATCAAA CTCAAAAGTA
CATGCTGGTC CCTTTGACTC CTTTCCACTA ACTGCCTTCT CCAAAGCAAT
CATTAAGCGA GCTGACCAAA CAGTGCTAAG TGTTCTTGTG
ATGACTTGAA ACCATCTATG CAAATCGATG ACACTAAGTG AGC
AGC6 reverse compliment: [663 bp]
GCTCACTTAG TGTCATCGAT TTGCATAGAT GGTTTCAAGT
CATCACAAGA ACACTTAGCA CTGTTTGGTC AGCTCGCTTA ATGATTGCTT
TGGAGAAGGC AGTTAGTGGA AAGGAGTCAA AGGGACCAGC
ATGTACTTTT GAGTTTGATG GTGAAAGCTC TGGTTTGCTL GGTCCAGGGG
AAAGCCGATG GCCGTTCACT AATGGGTATG CATTTGCAAC ATGGATTTAC
ATTGAATCAT TTGCTGATAC GTTAAATACA GCAACTGCTG
CTGCTGCAAT AGCTGCTGCT GCTGCTGCTA AGTCAGGAAA ATCTTCAGCT
ATGTCGGCTG CTGCTGCTGC CAGTGCGCTT GCAGGTGAAG
GAACAGCGCA TATGCCACGT CTGTTCAGTT TCTTGTCTGC TGACAACCAG
GGAATAGAGG CATACTTCCA TGCACAGTTT TTGGTGGTGG
AAAGTGGAAG CGGAAAGGGA AAGAAGGCTT CTTTACATTT
TACTCATGCA TTCAAGCCTC AGTGCTGGTA CTTCGTTGGT CTTGAGCATA
CTTGCAAACA AGGGCTGCTT GGGAAAGCAG AGAGTGAMCT
SAGATTGTAT ATTGCCAAGC TTCCCGGGTA CCGCAATCCC GCGGGCCATG
GCGGCCGGGA GCATGCGACG TCGGGCTCAW GTA

### Example 10

This example illustrates the amplicons produced during the amplification of STR locus AGC 8 with multiplex cocktails comprising primer pairs SEQ ID NO: 19 and SEQ ID NO: 20.

Sequence for AGC 8 locus:
GCGGTACCCGGGAAGCTTGGATCCCAAGATCCCCTACCTCTTTCGTTCTGAGG
CACGCCAGAAGATTTAGAAGTATCAATAGCTCCAAATTCAGAAGAGACACCT
CTGTTAACGGCGTGTCTAAGGTTCCCTTCCGACACCGGCGACGCACTCGAG
CTCCATACGAACATATGAAGGTCCTTGTTCGGCAGACCATTATTagcagcagcagca
gcaggaggaggTGCTGTAACAGTTGTTGCGTCTTTCTTCTTAACAGCCGTATTACTT
GTCGACCCGGAAAACATCGGATTAGGAGGAGGGTAAGACGGGGCAAGACCG
CCATTGAAGAGCTCTCCACTCATGCTCCTCGCTCCTCTCTGCTTCTTTCCCAT
ATTTTTCATCATCTCTTCGTCGAAATTAGATGTCCTTGGCGTGACGCCTTTC
GATGACTGAAGTGAGTAGACATCAGCGCCGTGAGTTGGTCCACCACCGTAGC
TGTTGGTGTACCCGTGTTTGGGACTAGCGGCCTTACTGGCATTAAACATGGCG
TAAAAATCAGTCTGGTTGAAGCTCGATGCCCTCGGGGTCGGCTCTCGCGAGG
ATTGTACAGAGTAGATCCCAAGCTTCCCGGGTACCGC
AGC8F: TTCCGACACC GGCGACGCAC TC [22 bp]
AGC8R: TTCTTTCCCA TATTTTTCAT CATCTCTTCG TCGAA [35 bp]
AGC8F (rev. comp.): GAGTGCGTCG CCGGTGTCGG AA [22 bp]
AGC8R (rev. comp.): TTCGACGAAG AGATGATGAA AAATATGGGA AAGAA
[35bp]
AGC8 array: AGCAGCAGCA GCAGCAGGAG GAGG [28 bp]
AGC8 motif: (AGC)5 + (AGG)3
AGC8 locus: [620 bp]
GCGGTACCCG GGAAGCTTGG ATCCCAAGAT CCCCTACCTC TTTCGTTCTG
AGGCACGCCA GAAGATTTAG AAGTATCAAT AGCTCCAAAT
TCAGAAGAGA CACCTCTGTT AACGGCGTGT CTAAGGTTCC CTTCCGACAC
CGGCGACGCA CTCGAGCTCC ATACGAACAT ATGAAGGTCC
TTGTTCGGCA GACCATTATT AGCAGCAGCA GCAGCAGGAG
GAGGTGCTGT AACAGTTGTT GCGTCTTTCT TCTTAACAGC CGTATTACTT
GTCGACCCGG AAAACATCGG ATTAGGAGGA GGGTAAGACG
GGGCAAGACC GCCATTGAAG AGCTCTCCAC TCATGCTCCT CGCTCCTCTC
TGCTTCTTTC CCATATTTTT CATCATCTCT TCGTCGAAAT TAGATGTCCT
TGGCGTGACG CCTTTCGATG ACTGAAGTGA GTAGACATCA
GCGCCGTGAG TTGGTCCACC ACCGTAGCTG TTGGTGTACC CGTGTTTGGG
ACTAGCGGCC TTACTGGCAT TAAACATGGC GTAAAAATCA
GTCTGGTTGA AGCTCGATGC CCTCGGGGTC GGCTCTCGCG AGGATTGTAC
AGAGTAGATC CCAAGCTTCC CGGGTACCGC
AGC8 reverse, compliment: [620 bp]
GCGGTACCCG GGAAGCTTGG GATCTACTCT GTACAATCCT
CGCGAGAGCC GACCCCGAGG GCATCGAGCT TCAACCAGAC
TGATTTTTAC GCCATGTTTA ATGCCAGTAA GGCCGCTAGT CCCAAACACG
GGTACACCAA CAGCTACGGT GGTGGACCAA CTCACGGCGC
TGATGTCTAC TCACTTCAGT CATCGAAAGG CGTCACGCCA AGGACATCTA
ATTTCGACGA AGAGATGATG AAAAATATGG GAAAGAAGCA
GAGAGGAGCG AGGAGCATGA GTGGAGAGCT CTTCAATGGC
GGTCTTGCCC CGTCTTACCC TCCTCCTAAT CCGATGTTTT CCGGGTCGAC
AAGTAATACG GCTGTTAAGA AGAAAGACGC AACAACTGTT
ACAGCACCTC CTCCTGCTGC TGCTGCTGCT AATAATGGTC TGCCGAACAA
GGACCTTCAT ATGTTCGTAT GGAGCTCGAG TGCGTCGCCG
GTGTCGGAAG GGAACCTTAG ACACGCCGTT AACAGAGGTG
TCTCTTCTGA ATTTGGAGCT ATTGATACTT CTAAATCTTC TGGCGTGCCT
CAGAACGAAA GAGGTAGGGG ATCTTGGGAT CCAAGCTTCC
CGGGTACCGC

### Example 11

This example illustrates the amplicons produced during the amplification of STR locus AGC 9 with multiplex cocktails comprising primer pairs SEQ ID NO: 21 and SEQ ID NO: 22.

Sequence for AGC 9 locus:
GCGGTACCCGGGAAGCTTGGTACACTCTACATGGCTCAAATTCTCCCGGTAA
GTTGATACATTCCTTCCCAGCATGGAAAACAGAGTAGCCagcagcagcagcagcag
cagcagcACGTCATATCAATCCAATTGCATTGTATTCTCCTTTAACTCATACAGCT
ATAGTTATGGCTGCCAACATATCTTCTCATCTCTTCCACTTAGCTTAATCAACT
CTCTTGGATACTAGGCAATTCGGTAACAGTTTACAAGTGTTAACCAGACGAC
AAAAAAAGAATTGTACACGTCCAGAATGGTGTCAGGGCCTACTAAAGGTTGA
ACCCAATTATTTTCTCAGGAATGGCTTTTGGCAAACAAGTAGCCTTTGGTCA
CTGCCATTCTGAAGATCCCAAGCTTCCCGGGTACCGC
AGC9F: GGTAAGTTGA TACATTCCTT CCC [23 bp]
AGC9R: CAAGTAGCCT TTGGTCACTG C [21 bp]
AGC9F (rev. comp.): GGGAAGGAAT GTATCAACTT ACC [23 bp]
AGC9R (rev. comp.): GCAGTGACCA AAGGCTACTT G [21 bp]
AGC9 array: AGCAGCAGCA GCAGCAGCAG CAGC [24 bp]
AGC9 motif: (AGCC)8
AGC9 locus: [411 bp]
GCGGTACCCG GGAAGCTTGG TACACTCTAC ATGGCTCAAA
TTCTCCCGGT AAGTTGATAC ATTCCTTCCC AGCATGGAAA ACAGAGTAGC
CAGCAGCAGC AGCAGCAGCA GCAGCACGTC ATATCAATCC
AATTGCATTG TATTCTCCTT TAACTCATAC AGCTATAGTT ATGGCTGCCA
ACATATCTTC TCATCTCTTC CACTTAGCTT AATCAACTCT CTTGGATACT
AGGCAATTCG GTAACAGTTT ACAAGTGTTA ACCAGACGAC
AAAAAAAGAA TTGTACACGT CCAGAATGGT GTCAGGGCCT
ACTAAAGGTT GAACCCAATT ATTTTCTCAG GAATGGCTTT TGGCAAACAA
GTAGCCTTTG GTCACTGCCA TTCTGAAGAT CCCAAGCTTC CCGGGTACCG
C
AGC9 reverse compliment: [411 bp]
GCGGTACCCG GGAAGCTTGG GATCTTCAGA ATGGCAGTGA
CCAAAGGCTA CTTGTTTGCC AAAAGCCATT CCTGAGAAAA TAATTGGGTT
CAACCTTTAG TAGGCCCTGA CACCATTCTG GACGTGTACA ATTCTTTTTT
TGTCGTCTGG TTAACACTTG TAAACTGTTA CCGAATTGCC TAGTATCCAA
GAGAGTTGAT TAAGCTAAGT GGAAGAGATG AGAAGATATG
TTGGCAGCCA TAACTATAGC TGTATGAGTT AAAGGAGAAT
ACAATGCAAT TGGATTGATA TGACGTGCTG CTGCTGCTGC TGCTGCTGCT
GGCTACTCTG TTTTCCATGC TGGGAAGGAA TGTATCAACT TACCGGGAGA
ATTTGAGCCA TGTAGAGTGT ACCAAGCTTC CCGGGTACCG C

### Example 12

This example illustrates the amplicons produced during the amplification of STR locus AGC 10 with multiplex cocktails comprising primer pairs SEQ ID NO: 23 and SEQ ID NO: 24.

Sequence for AGC 10 locus:
GCGGTACCCGGGAAGCTTGGATCAGCGGCAACAACAAcagcaacaacaacatcagca
gcagcagcaacaacaacaacatcagcagcagcagcagcagcagcagcagcagcatcaacatcagcaacagcagca
acagcagcagcagcagcagcagcagcaacagcagcagcaacagcagcagcaacaacaccagcatcagcaacacca
gcagcagcaacaccagcatcagcagcaacatcagcagcagcagcTTCAACCGTCACAACAATTGCA
TCAGTTGTCTGTTCAGCAGCAGATTCCTAATGTTATGTCTGCTCTACCCAGT
TTTTCCTCTGGTACTCAGTCTCAGTCTCCATCGCTGCAGGCCATCCCTTCACA
GTGCCAGCAGCCAAGCTTCCCGGGTACCGC
AGC10F: GGATCAGCGG CAACAACAA [19 bp]
AGC10R: TGTTATGTCT GCTCTACCCA GTTTT [25 bp]
AGC10F (rev. comp.): TTGTTGTTGC CGCTGATCC [19 bp]
AGC10R (rev, camp.): AAAACTGGGT AGAGCAGACA TAACA [25 bp]
AGC10 array: AGCAACAACA ACATCAGCAG CAGCAGCAAC AACAACAACA
TCAGCAGCAG CAGCAGCAGC AGCAGCAGCA GCATCAACAT
CAGCAACAGC AGCAACAGCA GCAGCAGCAG CAGCAGCAGC
AACAGCAGCA GCAACAGCAG CAGCAACAAC ACCAGCATCA
GCAACACCAG CAGCAGCAAC ACCAGCATCA GCAGCAACAT
CAGCAGCAGC AGC [213 bp]
AGC10 motif: (AGC)1 + (AAC)3 + (ATC)1 + (AGC)4 + (AAC)4 + (ATC)1 + (AGC)10 +
(ATC)1 + (AACATC)1 + (AGCAAC)1 + (AGC)2 + (AAC)1 + (AGC)8 + (AAC)1 +
(AGC)3 + (AAC)1 + (AGC)3 + (AAC)2 + (ACC)1 + (AGC)1 + (ATC)1 + (AGC)1 +
(AACACC)1 + (AGC)3 + (AACACC)1 + (AGC)3 + (AACACC)1 + (AGC)3 +
(AACACC)1 + (AGCATC)1 + (AGC)2 + (AACATC)1 + (AGC)4
AGC10 locus: [408 bp]
GCGGTACCCG GGAAGCTTGG ATCAGCGGCA ACAACAACAG
CAACAACAAC ATCAGCAGCA GCAGCAACAA CAACAACATC
AGCAGCAGCA GCAGCAGCAG CAGCAGCAGC ATCAACATCA
GCAACAGCAG CAACAGCAGC AGCAGCAGCA GCAGCAGCAA
CAGCAGCAGC AACAGCAGCA GCAACAACAC CAGCATCAGC
AACACCAGCA GCAGCAACAC CAGCATCAGC AGCAACATCA
GCAGCAGCAG CTTCAACCGT CACAACAATT GCATCAGTTG TCTGTTCAGC
AGCAGATTCC TAATGTTATG TCTGCTCTAC CCAGTTTTTC CTCTGGTACT
CAGTCTCAGT CTCCATCGCT GCAGGCCATC CCTTCACAGT GCCAGCAGCC
AAGCTTCCCG GGTACCGC
AGC10 reverse compliment: [408 bp]
GCGGTACCCG GGAAGCTTGG CTGCTGGCAC TGTGAAGGGA
TGGCCTGCAG CGATGGAGAC TGAGACTGAG TACCAGAGGA
AAAACTGGGT AGAGCAGACA TAACATTAGG AATCTGCTGC
TGAACAGACA ACTGATGCAA TTGTTGTGAC GGTTGAAGCT
GCTGCTGCTG ATGTTGCTGC TGATGCTGGT GTTGCTGCTG CTGGTGTTGC
TGATGCTGGT GTTGTTGCTG CTGCTGTTGC TGCTGCTGTT GCTGCTGCTG
CTGCTGCTGC TGCTGTTGCT GCTGTTGCTG ATGTTGATGC TGCTGCTGCT
GCTGCTGCTG CTGCTGCTGA TGTTGTTGTT GTTGCTGCTG CTGCTGATGT
TGTTGTTGCT GTTGTTGTTG CCGCTGATCC AAGCTTCCCG GGTACCGC

### Example 13

This example illustrates the amplicons produced during the amplification of STR locus ACT 1 with multiplex cocktails comprising primer pairs SEQ ID NO: 25 and SEQ ID NO: 26.

Sequence for ACT 1 locus:
GCGGTACCCGGGAAGCTTGGGATCAAAAAACGAGAAGAATATTCATCATGA
AAAACTCTATAGAACTTTTATTATTCAAAGTAGGAAGGAACAAGGAAGAGGG
AAGAAAAAAAAAGAAGGGGGCAGAGGGGGGCAATTTATGTTTGCCTTTTATG
CTATATATTTTAGTATCTAGAAGAACAAGAAAAAAAGACTATACTCCTAATA
TGAATATGGAACTAAAAAATTGACTCAGCATATTAAAGCAGAAACTTTGAA
ATAGACGAACCATGTTTTGGTTTACAACTGTGGTTTTTGTATTGACATCTAGT
TGTAAGGAactactactactactACCTGTGCAAAAGGTGAACTCTCTACCATGAAAGT
AGTAATGGTTTTCAAGGGCCATTTAACTTGAACCACCATAGCTAGCAAAGGT
GGTTTACATATTCCACTTGTTTGTGAGCCACGCAAAGTGAGTTCCTATTAA
CCAGTTTTAAAACATATGTCATTTCCAAGATAGTTGAAAACCTCGGAAGCAG
CAGCATTACTGTTTTTCATAGCATTTCCAGGATTGTTGAAAACTTCAGCAGCA
GCAGCAGCAGCAACAGTATTACTGTTTTTTATAGCATCTCCATTTTGGTTCAC
AGTGAAATCCACAGTAAAGGAATTTAGACT
ACT1F: GACTCAGCAT ATTAAAGCAG AAACT [25 bp]
ACT1R: GTTTACATAT TCCACTTGTT TGTGA [25 bp]
ACT1F (rev. comp.): AGTTTCTGCT TTAATATGCT GAGTC [25 bp]
ACT1R (rev. comp.): TCACAAACAA GTGGAATATG TAAAC [25 bp]
ACT1 array: ACTACTACTA CTACT [15 bp]
ACT1 motif: (ACT)5
ACT1 locus: [660 bp]
GCGGTACCCG GGAAGCTTGG GATCAAAAAA CGAGAAGAAT
ATTCATCATG AAAAACTCTA TAGAACTTTT ATTATTCAAA GTAGGAAGGA
ACAAGGAAGA GGGAAGAAAA AAAAAGAAGG GGGCAGAGGG
GGGCAATTTA TGTTTGCCTT TTATGCTATA TATTTTAGTA TCTAGAAGAA
CAAGAAAAAA AGACTATACT CCTAATATGA ATATGGAACT
AAAAAATTGA CTCAGCATAT TAAAGCAGAA ACTTTGAAAT
AGACGAACCA TGTTTTGGTT TACAACTGTG GTTTTTGTAT TGACATCTAG
TTGTAAGGAA CTACTACTAC TACTACCTGT GCAAAAGGTG AACTCTCTAC
CATGAAAGTA GTAATGGTTT TCAAGGGCCA TTTAACTTGA
ACCACCATAG CTAGCAAAGG TGGTTTACAT ATTCCACTTG TTTGTGAGCC
ACGCAAAGTG AGTTCCTATT AACCAGTTTT AAAACATATG TCATTTCCAA
GATAGTTGAA AACCTCGGAA GCAGCAGCAT TACTGTTTTT CATAGCATTT
CCAGGATTGT TGAAAACTTC AGCAGCAGCA GCAGCAGCAA
CAGTATTACT GTTTTTTATA GCATCTCCAT TTTGGTTCAC AGTGAAATCC
ACAGTAAAGG AATTTAGACT
ACT1 reverse compliment: [660 bp]
AGTCTAAATT CCTTTACTGT GGATTTCACT GTGAACCAAA ATGGAGATGC
TATAAAAAAC AGTAATACTG TTGCTGCTGC TGCTGCTGCT GAAGTTTTCA
ACAATCCTGG AAATGCTATG AAAAACAGTA ATGCTGCTGC
TTCCGAGGTT TTCAACTATC TTGGAAATGA CATATGTTTT AAAACTGGTT
AATAGGAACT CACTTTGCGT GGCTCACAAA CAAGTGGAAT
ATGTAAACCA CCTTTGCTAG CTATGGTGGT TCAAGTTAAA TGGCCCTTGA
AAACCATTAC TACTTTCATG GTAGAGAGTT CACCTTTTGC ACAGGTAGTA
GTAGTAGTAG TTCCTTACAA CTAGATGTCA ATACAAAAAC
CACAGTTGTA AACCAAAACA TGGTTCGTCT ATTTCAAAGT TTCTGCTTTA
ATATGCTGAG TCAATTTTTT AGTTCCATAT TCATATTAGG AGTATAGTCT
TTTTTTCTTG TTCTTCTAGA TACTAAAATA TATAGCATAA AAGGCAAACA
TAAATTGCCC CCCTCTGCCC CCTTCTTTTT TTTTCTTCCC TCTTCCTTGT
TCCTTCCTAC TTTGAATAAT AAAAGTTCTA TAGAGTTTTT CATGATGAAT
ATTCTTCTCG TTTTTTGATC CCAAGCTTCC CGGGTACCGC

### Example 14

This example illustrates the amplicons produced during the amplification of STR locus CCT 2 with multiplex cocktails comprising primer pairs SEQ ID NO: 2 and SEQ ID NO: 28.

Sequence for CCT 2 locus:
GCGGTACCCGGGAAGCTTGGGATCGTGCAGTGGATGTGTCGGGTTCGAAA
GTCTATcctcctcctcctcctGCCGTTGGA
ATGGTGTGTTCGTCTCTGCCTGTTCAAAGAGCGACAATCAATGGTCTTAAAGG
AGCACCTATCTGCCTGACTGGAAATCCAAGCTCCCTCCGATGAATGATTGTTT
GTTCTTGCTTGATTACCGGAGGACCGACGCAGGAAGGCGTTGTCACTGCGAC
TTGGTGCCTACTATGCTCTTCACGGAAAGGAGTGAAACGAGCAAGGAGAGAG
TCAACCTTAATGTCAGTGATAATAGTAAAGGAAGAGACAGAATCTCATCTGC
TTGGCTGGTCGACACAAGCAATGCCCAAAGAGCATTCTTTTCTATTTTCATGC
TTCATAATGTATCCGCCGGATTGAAACAGTCTCTTTTGTGCCTGACCTAATC
CTCTAGCTCTTTACTTGCCAGGAGAAGGCTCGCCAAGCTTCCCGGGTACCGC
CCT2F: GCAGTGGATG TGTCGGGT [18 bp]
CCT2R: TTTGTGCCTG ACCTAATCCT CTA [23 bp]
CCT2F (rev. comp.): ACCCGACACA TCCACTGC [18 bp]
CCT2R (rev. comp.): TAGAGGATTA GGTCAGGCAC AAA [23 bp]
CCT2 array: CCTCCTCCTC CTCCT [15 bp]
CCT2 motif: (CCT)5
CCT2 locus: [499 bp]
GCGGTACCCG GGAAGCTTGG GATCGTGCAG TGGATGTGTC
GGGTTCGAAA GTCTATCCTC CTCCTCCTCC TGCCGTTGGA ATGGTGTGTT
CGTCTCTGCC TGTTCAAAGA GCGACAATCA ATGGTCTTAA
AGGAGCACCT ATCTGCCTGA CTGGAAATCC AAGCTCCCTC
CGATGAATGA TTGTTTGTTC TTGCTTGATT ACCGGAGGAC CGACGCAGGA
AGGCGTTGTC ACTGCGACTT GGTGCCTACT ATGCTCTTCA CGGAAAGGAG
TGAAACGAGC AAGGAGAGAG TCAACCTTAA TGTCAGTGAT
AATAGTAAAG GAAGAGACAG AATCTCATCT GCTTGGCTGG
TCGACACAAG CAATGCCCAA AGAGCATTCT TTTCTATTTT CATGCTTCAT
AATGTATCCG CCGGATTGAA ACAGTCTCTT TTGTGCCTGA CCTAATCCTC
TAGCTCTTTA CTTGCCAGGA GAAGGCTCGC CAAGCTTCCC GGGTACCGC
CCT2 locus reverse compliment: [499 bp]
GCGGTACCCG GGAAGCTTGG CGAGCCTTCT CCTGGCAAGT
AAAGAGCTAG AGGATTAGGT CAGGCACAAA AGAGACTGTT
TCAATCCGGC GGATACATTA TGAAGCATGA AAATAGAAAA
GAATGCTCTT TGGGCATTGC TTGTGTCGAC CAGCCAAGCA GATGAGATTC
TGTCTCTTCC TTTACTATTA TCACTGACAT TAAGGTTGAC TCTCTCCTTG
CTCGTTTCAC TCCTTTCCGT GAAGAGCATA GTAGGCACCA AGTCGCAGTG
ACAACGCCTT CCTGCGTCGG TCCTCCGGTA ATCAAGCAAG
AACAAACAAT CATTCATCGG AGGGAGCTTG GATTTCCAGT
CAGGCAGATA GGTGCTCCTT TAAGACCATT GATTGTCGCT CTTTGAACAG
GCAGAGACGA ACACACCATT CCAACGGCAG GAGGAGGAGG
AGGATAGACT TTCGAACCCG ACACATCCAC TGCACGATCC
CAAGCTTCCC GGGTACCGC

While certain of the preferred embodiments of the present invention have been described and specifically exemplified above, it is not intended that the invention be limited to such embodiments. Various modifications may be made thereto without departing from the scope and spirit of the present invention, as set forth in the following claims.

TABLE 1
Collection of worldwide samples with representatives
from all continents except Australia.

| Continent | # of Samples |
|---|---|
| North America | |
| U.S.A. | 188 |
| Canada | 1 |
| Mexico | 7 |
| Total North America | 196 |
| Central & South America | |
| Colombia | 3 |
| Costa Rica | 6 |
| Jamaica | 4 |
| Total C & S America | 13 |
| Africa | |
| Nigeria | 1 |
| South Africa | 6 |
| Sierra Leone | 2 |
| Uganda | 2 |
| Zimbabwe | 2 |
| Total Africa | 13 |
| Asia | |
| Afghanistan | 14 |
| Cambodia | 1 |
| China | 4 |
| India | 5 |
| Japan | 3 |
| Korea | 4 |
| Kurdistan | 2 |
| Nepal | 1 |
| Pakistan | 2 |
| Russia | 4 |
| Thailand | 1 |
| Turkey | 3 |
| Uzbekistan | 2 |
| Total Asia | 46 |
| Europe | |
| Czechoslovakia | 1 |
| France | 3 |
| Germany | 4 |
| Holland | 2 |
| Hungary | 8 |
| Italy | 3 |
| Poland | 3 |
| Romania | 1 |
| Spain | 2 |
| Total Europe | 27 |
| Total # Samples = | 295 |

TABLE 2

Attributes of eight microsatellite loci developed for Cannabis sativa. Values in the
‘Amplicon Size Range (bp)’ refer to results from fragment analyses of 295 C. sativa
samples. ‘Number of Alleles’ reflects the number of alleles observed in this data set.

| | | | | | | |
|---|---|---|---|---|---|---|
| Locus | | | Amplicon | | | |
| Name | | | Size | | Number | |
| Dye | | Repeat | Range | Tₘ | of | |
| Label^{b} | Primer Sequences | Motifs^{b} | (bp) | (°C.) | Alleles | H_{F.} |

| | | | | | | |
|---|---|---|---|---|---|---|
| AAAG1 | F: 5′GTCAGAAAGCGAAGACCTTTAGA 3′ | (AAAG)₆ | 103-135 | 59 | 16 | 0.684 |
| HEX | R: 5′GATGATGCCTGCCTGTCTTTAC 3′ | | | | | |
| AAAG5 | F: 5′GTCAATTAATGCTTATAGCCCATATGTTTTCTACTAC 3′ | (AAAG)₅ | 188-200 | 59 | 4 | 0.625 |
| NED | R: 5′GCAACTTCAGGAATACTTTGTTTCTTCTTGTTCT 3′ | | | | | |
| AGC1 | F: 5′GCAAAGAGTGTATCGAAACCTGTC 3′ | (AGC)₁₀ | 128-164 | 59 | 10 | 0.656 |
| FAM | R: 5′GCCCACCACATCGTCTGTATTAGTAC 3′ | | | | | |
| AGC6 | F: 5′GAGACGTGGCATATGCGCTGTTCCTTCA 3′ | (AGC)₆ | 200 & | 62 | 2 | 0.132 |
| HEX | R: 5′GCCGATGGCCGTTCACTAATGGGTATGC 3′ | | 221 | | | |
| AGC8 | F: 5′GTTCCGACACCGGCGACGCACTC 3′ | (AGC)₅ | 264-279 | 59 | 6 | 0.591 |
| NED & | R: 5′GTTCGACGAAGAGATGATGAAAAATATGGGAAAGAA 3′ | | | | | |
| FAM | | | | | | |
| AGC9 | F: 5′GGTAAGTTGATACATTCCTTCCC 3′ | (AGC)₉ | 317-335 | 62 | 7 | 0.698 |
| HEX | R: 5′GCAGTGACCAAAGGCTACTTG 3′ | | | | | |
| AGC10 | F: 5′GGATCAGCGGCAACAACAA 3′ | (AGC)₄₃ | 273-327 | 62 | 15 | 0.776 |
| NED | R: 5′GAAAACTGGGTAGAGCAGACATAACA 3′ | | | | | |
| ACT1 | F: 5′GACTCAGCATATTAAAGCAGAAACT 3′ | (ACT)₆ | 218-224 | 59 | 3 | 0.440 |
| FAM | R: 5′GTCACAAACAAGTGGAATATGTAAAC 3′ | | | | | |

^{a}HEX & FAM labeled primers were ordered from Integrative DNA Technologies; NED labeled primers were orcered from Perken Elmer
^{b}Most repeat motifs are not perfect and appear to be complete

### APPENDIX 1: Raw STR Data

Allelic scores, in base pairs, for all 295 samples genotyped across eight polymorphic loci. Samples where the same allelic size is listed twice are homozygous, whereas two different allelic sizes indicate a heterozygous state. Marker names are displayed across the top row of each page.

| Sample | AAAG1 | | ACT1 | | AGC8 | | AGC9 | | AGC1 | | AAAG5 | | AGC6 | | AGC10 | |
|---|---|---|---|---|---|---|---|---|---|---|---|---|---|---|---|---|
| AFG177 | 127 | 127 | 221 | 221 | 264 | 270 | 326 | 326 | 152 | 152 | 192 | 192 | 200 | 200 | 309 | 321 |
| AFG178 | 127 | 127 | 221 | 221 | 264 | 270 | 326 | 326 | 140 | 152 | 192 | 192 | 200 | 200 | 321 | 321 |
| AFG181 | 103 | 117 | 221 | 221 | 270 | 270 | 326 | 326 | 140 | 152 | 196 | 196 | 200 | 200 | 294 | 303 |
| AFG182 | 103 | 117 | 221 | 221 | 270 | 270 | 326 | 326 | 140 | 152 | 196 | 196 | 200 | 200 | 306 | 306 |
| AFG217 | 117 | 117 | 218 | 221 | 264 | 264 | 326 | 326 | 152 | 164 | 192 | 192 | 200 | 200 | 303 | 309 |
| AFG218 | 117 | 123 | 218 | 218 | 264 | 270 | 326 | 326 | 152 | 152 | 192 | 192 | 200 | 200 | 309 | 309 |
| AFG223 | 117 | 127 | 218 | 221 | 270 | 270 | 332 | 332 | 131 | 131 | 188 | 196 | 200 | 200 | 309 | 309 |
| AFG224 | 117 | 127 | 218 | 221 | 270 | 270 | 320 | 326 | 137 | 152 | 196 | 196 | 200 | 200 | 300 | 300 |
| AFG225 | 127 | 127 | 221 | 221 | 267 | 267 | 320 | 323 | 140 | 152 | 188 | 192 | 200 | 200 | 300 | 309 |
| AFG61 | 123 | 127 | 221 | 221 | 270 | 270 | 326 | 329 | 164 | 164 | 188 | 192 | 200 | 200 | 300 | 309 |
| AFG62 | 117 | 127 | 218 | 221 | 270 | 270 | 326 | 326 | 131 | 131 | 192 | 192 | 200 | 221 | 300 | 300 |
| AFG63 | 117 | 117 | 218 | 218 | 270 | 270 | 326 | 332 | 152 | 164 | 192 | 200 | 200 | 200 | 300 | 309 |
| AFG64 | 127 | 127 | 218 | 218 | 270 | 270 | 326 | 326 | 152 | 164 | 192 | 192 | 200 | 200 | 309 | 324 |
| AFG83 | 127 | 127 | 221 | 221 | 270 | 276 | 326 | 326 | 152 | 152 | 196 | 196 | 200 | 200 | 309 | 312 |
| AK81 | 117 | 127 | 218 | 221 | 270 | 270 | 326 | 332 | 152 | 152 | 188 | 188 | 200 | 200 | 300 | 300 |
| AK82 | 117 | 127 | 221 | 221 | 270 | 270 | 329 | 332 | 152 | 152 | 188 | 188 | 200 | 200 | 300 | 315 |
| AZ100 | 117 | 127 | 218 | 218 | 270 | 276 | 323 | 332 | 131 | 152 | 196 | 200 | 200 | 221 | 309 | 315 |
| AZ101 | 117 | 127 | 218 | 221 | 270 | 276 | 326 | 326 | 131 | 131 | 188 | 200 | 200 | 221 | 309 | 315 |
| AZ102 | 117 | 123 | 221 | 221 | 264 | 270 | 323 | 332 | 131 | 152 | 188 | 200 | 200 | 200 | 315 | 315 |
| AZ103 | 117 | 127 | 218 | 218 | 270 | 270 | 326 | 332 | 140 | 140 | 188 | 192 | 200 | 200 | 315 | 324 |
| AZ104 | 117 | 117 | 218 | 221 | 270 | 276 | 323 | 332 | 131 | 152 | 192 | 200 | 200 | 200 | 309 | 315 |
| AZ176 | 127 | 127 | 218 | 218 | 264 | 270 | 326 | 332 | 140 | 140 | 192 | 196 | 200 | 200 | 300 | 309 |
| AZ97 | 117 | 123 | 221 | 221 | 264 | 270 | 323 | 332 | 131 | 152 | 188 | 200 | 200 | 200 | 315 | 315 |
| AZ98 | 117 | 117 | 221 | 221 | 270 | 270 | 323 | 332 | 131 | 152 | 192 | 200 | 200 | 200 | 315 | 315 |
| AZ99 | 127 | 127 | 218 | 221 | 264 | 276 | 326 | 326 | 152 | 152 | 192 | 196 | 221 | 221 | 309 | 309 |
| CA121 | 117 | 127 | 221 | 221 | 264 | 276 | 323 | 329 | 137 | 152 | 188 | 192 | 200 | 200 | 315 | 321 |
| CA122 | 117 | 117 | 221 | 221 | 264 | 270 | 326 | 329 | 137 | 152 | 192 | 192 | 200 | 200 | 312 | 321 |
| CA123 | 117 | 117 | 221 | 221 | 264 | 270 | 323 | 323 | 137 | 152 | 192 | 192 | 200 | 200 | 309 | 315 |
| CA124 | 121 | 123 | 221 | 221 | 264 | 264 | 323 | 326 | 152 | 152 | 192 | 192 | 200 | 200 | 306 | 309 |
| CA125 | 123 | 127 | 218 | 221 | 264 | 270 | 326 | 329 | 152 | 152 | 192 | 192 | 200 | 200 | 306 | 306 |
| CA126 | 127 | 127 | 218 | 221 | 264 | 270 | 326 | 332 | 131 | 152 | 192 | 196 | 200 | 200 | 312 | 315 |
| CA127 | 127 | 127 | 218 | 221 | 264 | 270 | 326 | 332 | 131 | 152 | 192 | 196 | 200 | 200 | 312 | 315 |
| CA128 | 117 | 117 | 224 | 224 | 270 | 270 | 326 | 326 | 140 | 152 | 188 | 188 | 200 | 200 | 300 | 300 |
| CA129 | 117 | 117 | 221 | 221 | 270 | 270 | 326 | 326 | 140 | 152 | 188 | 188 | 200 | 200 | 300 | 300 |
| CA130 | 113 | 123 | 221 | 221 | 264 | 264 | 326 | 329 | 152 | 152 | 192 | 192 | 200 | 200 | 309 | 315 |
| CA131 | 113 | 123 | 221 | 221 | 264 | 264 | 326 | 329 | 152 | 152 | 188 | 192 | 200 | 200 | 309 | 315 |
| CA132 | 127 | 127 | 218 | 218 | 270 | 279 | 326 | 326 | 152 | 152 | 188 | 192 | 200 | 200 | 309 | 309 |
| CA133 | 113 | 117 | 218 | 221 | 264 | 264 | 329 | 329 | 152 | 152 | 192 | 192 | 200 | 200 | 315 | 324 |
| CA134 | 123 | 127 | 221 | 221 | 270 | 270 | 326 | 326 | 152 | 152 | 192 | 192 | 200 | 200 | 309 | 309 |
| CA135 | 127 | 127 | 218 | 221 | 264 | 264 | 326 | 326 | 152 | 152 | 192 | 200 | 200 | 200 | 309 | 309 |
| CA136 | 117 | 117 | 221 | 221 | 264 | 270 | 326 | 326 | 152 | 152 | 188 | 192 | 200 | 200 | 309 | 312 |
| CA137 | 117 | 117 | 221 | 221 | 270 | 270 | 323 | 329 | 152 | 152 | 192 | 192 | 200 | 200 | 309 | 321 |
| CA138 | 117 | 117 | 221 | 221 | 270 | 270 | 326 | 326 | 152 | 152 | 188 | 192 | 200 | 200 | 309 | 309 |
| CA139 | 117 | 117 | 221 | 221 | 264 | 270 | 326 | 326 | 152 | 152 | 192 | 192 | 200 | 200 | 309 | 309 |
| CA140 | 117 | 117 | 218 | 221 | 264 | 270 | 323 | 326 | 146 | 152 | 192 | 192 | 200 | 200 | 312 | 315 |
| CA141 | 117 | 117 | 221 | 221 | 264 | 270 | 323 | 326 | 152 | 152 | 192 | 192 | 200 | 200 | 300 | 309 |
| CA142 | 117 | 117 | 221 | 221 | 264 | 270 | 323 | 326 | 152 | 152 | 192 | 192 | 200 | 200 | 300 | 309 |
| CA143 | 117 | 117 | 218 | 221 | 264 | 264 | 326 | 329 | 152 | 152 | 188 | 188 | 200 | 200 | 309 | 315 |
| CA144 | 117 | 117 | 218 | 221 | 264 | 264 | 326 | 329 | 152 | 152 | 188 | 188 | 200 | 200 | 309 | 315 |
| CA145 | 117 | 121 | 221 | 221 | 264 | 270 | 323 | 326 | 137 | 152 | 192 | 192 | 200 | 200 | 309 | 309 |
| CA146 | 117 | 121 | 221 | 221 | 264 | 270 | 323 | 326 | 137 | 152 | 192 | 192 | 200 | 200 | 309 | 309 |
| CA147 | 117 | 117 | 218 | 221 | 264 | 264 | 326 | 329 | 152 | 152 | 188 | 188 | 200 | 200 | 309 | 315 |
| CA148 | 117 | 117 | 218 | 221 | 270 | 270 | 326 | 326 | 140 | 152 | 192 | 196 | 200 | 200 | 300 | 309 |
| CA149 | 127 | 127 | 221 | 221 | 264 | 264 | 326 | 326 | 152 | 152 | 188 | 188 | 200 | 200 | 300 | 318 |
| CA150 | 117 | 117 | 218 | 221 | 264 | 264 | 320 | 329 | 152 | 152 | 192 | 192 | 200 | 200 | 288 | 309 |
| CA72 | 117 | 127 | 221 | 221 | 270 | 270 | 323 | 326 | 152 | 152 | 188 | 192 | 200 | 200 | 300 | 300 |
| CA73 | 117 | 127 | 221 | 221 | 270 | 270 | 326 | 326 | 152 | 152 | 188 | 188 | 200 | 200 | 309 | 324 |
| CAM243 | 123 | 123 | 221 | 221 | 264 | 270 | 326 | 326 | 152 | 152 | 192 | 192 | 200 | 200 | 309 | 309 |
| CAN231 | 117 | 117 | 218 | 218 | 264 | 270 | 320 | 329 | 146 | 146 | 192 | 192 | 200 | 200 | 297 | 306 |
| CHI183 | 107 | 123 | 218 | 221 | 264 | 276 | 326 | 326 | 137 | 137 | 192 | 192 | 200 | 200 | 297 | 300 |
| CHI184 | 117 | 119 | 218 | 218 | 270 | 270 | 326 | 326 | 137 | 137 | 192 | 192 | 200 | 200 | 297 | 321 |
| CHI185 | 117 | 117 | 218 | 221 | 270 | 270 | 326 | 326 | 137 | 152 | 192 | 192 | 200 | 200 | 303 | 309 |
| CHI201 | 111 | 123 | 221 | 221 | 270 | 279 | 320 | 326 | 146 | 152 | 196 | 200 | 200 | 200 | 297 | 303 |
| COL67 | 117 | 117 | 221 | 221 | 264 | 279 | 323 | 326 | 152 | 152 | 188 | 188 | 200 | 200 | 309 | 309 |
| COL68 | 117 | 117 | 221 | 221 | 264 | 273 | 326 | 329 | 131 | 164 | 188 | 192 | 200 | 221 | 303 | 315 |
| COL69 | 117 | 117 | 221 | 224 | 279 | 279 | 323 | 326 | 152 | 152 | 188 | 192 | 200 | 200 | 309 | 309 |
| CoR170 | 117 | 117 | 218 | 221 | 279 | 279 | 323 | 326 | 164 | 164 | 188 | 192 | 200 | 200 | 309 | 309 |
| CoR171 | 117 | 117 | 221 | 221 | 270 | 273 | 323 | 323 | 164 | 164 | 188 | 188 | 200 | 200 | 309 | 309 |
| CoR172 | 117 | 117 | 218 | 221 | 270 | 279 | 323 | 323 | 146 | 152 | 192 | 192 | 200 | 200 | 309 | 309 |
| CoR173 | 117 | 117 | 218 | 218 | 264 | 279 | 323 | 326 | 146 | 152 | 188 | 192 | 200 | 200 | 309 | 309 |
| CoR174 | 117 | 117 | 218 | 221 | 270 | 273 | 323 | 323 | 152 | 152 | 188 | 188 | 200 | 200 | 309 | 309 |
| CoR175 | 117 | 117 | 218 | 218 | 270 | 270 | 323 | 326 | 152 | 152 | 188 | 188 | 200 | 200 | 309 | 309 |
| CT1 | 117 | 117 | 221 | 221 | 264 | 270 | 323 | 326 | 140 | 152 | 188 | 196 | 200 | 200 | 300 | 309 |
| CT2 | 117 | 117 | 221 | 221 | 264 | 270 | 323 | 326 | 140 | 152 | 188 | 196 | 200 | 200 | 300 | 309 |
| CT3 | 117 | 123 | 221 | 221 | 264 | 264 | 326 | 326 | 140 | 140 | 188 | 188 | 200 | 200 | 300 | 300 |
| CT4 | 117 | 117 | 218 | 221 | 264 | 270 | 326 | 332 | 152 | 152 | 188 | 188 | 200 | 200 | 300 | 309 |
| CT5 | 117 | 123 | 221 | 221 | 264 | 270 | 326 | 329 | 140 | 152 | 188 | 192 | 200 | 200 | 300 | 315 |
| CT6 | 117 | 123 | 221 | 221 | 264 | 270 | 326 | 329 | 140 | 152 | 188 | 192 | 200 | 200 | 300 | 315 |
| CT7 | 117 | 123 | 221 | 221 | 264 | 264 | 326 | 326 | 140 | 140 | 188 | 188 | 200 | 200 | 300 | 300 |
| CT8 | 117 | 117 | 221 | 221 | 264 | 264 | 326 | 326 | 140 | 152 | 188 | 188 | 200 | 200 | 300 | 300 |
| CT9 | 117 | 123 | 218 | 221 | 264 | 270 | 326 | 326 | 140 | 152 | 188 | 192 | 200 | 200 | 300 | 309 |
| CT10 | 117 | 127 | 221 | 221 | 264 | 270 | 326 | 326 | 152 | 152 | 188 | 192 | 200 | 221 | 300 | 300 |
| CT11 | 117 | 117 | 218 | 221 | 270 | 270 | 326 | 326 | 140 | 152 | 192 | 192 | 200 | 200 | 309 | 309 |
| CT12 | 117 | 117 | 221 | 221 | 264 | 279 | 332 | 332 | 140 | 152 | 188 | 188 | 200 | 200 | 309 | 309 |
| CT13 | 117 | 117 | 221 | 221 | 264 | 279 | 332 | 332 | 140 | 152 | 188 | 188 | 200 | 200 | 309 | 309 |
| CT14 | 123 | 123 | 221 | 221 | 270 | 270 | 326 | 332 | 140 | 152 | 188 | 188 | 200 | 200 | 309 | 309 |
| CT15 | 117 | 127 | 221 | 221 | 264 | 270 | 326 | 326 | 152 | 152 | 188 | 192 | 200 | 221 | 300 | 300 |
| CT16 | 117 | 123 | 221 | 221 | 264 | 264 | 326 | 326 | 140 | 140 | 188 | 188 | 200 | 200 | 300 | 300 |
| CT17 | 117 | 123 | 221 | 221 | 264 | 264 | 326 | 326 | 140 | 140 | 188 | 188 | 200 | 200 | 300 | 300 |
| CT18 | 117 | 127 | 221 | 221 | 264 | 270 | 326 | 326 | 152 | 152 | 188 | 192 | 200 | 221 | 300 | 300 |
| CT19 | 123 | 123 | 221 | 221 | 270 | 270 | 326 | 332 | 140 | 152 | 188 | 188 | 200 | 200 | 309 | 309 |
| CT20 | 117 | 127 | 221 | 221 | 264 | 270 | 326 | 326 | 152 | 152 | 188 | 192 | 200 | 221 | 300 | 300 |
| CT21 | 117 | 127 | 218 | 221 | 264 | 264 | 326 | 326 | 140 | 152 | 188 | 192 | 200 | 200 | 300 | 309 |
| CT22 | 117 | 127 | 221 | 221 | 264 | 273 | 326 | 326 | 131 | 152 | 188 | 196 | 200 | 200 | 300 | 321 |
| CT23 | 117 | 117 | 221 | 221 | 264 | 264 | 323 | 332 | 131 | 140 | 192 | 192 | 200 | 200 | 309 | 309 |
| CT24 | 117 | 117 | 221 | 221 | 270 | 270 | 326 | 326 | 152 | 152 | 188 | 188 | 200 | 200 | 309 | 309 |
| CT25 | 117 | 127 | 221 | 221 | 264 | 264 | 326 | 332 | 152 | 152 | 196 | 196 | 200 | 200 | 309 | 321 |
| CT26 | 117 | 127 | 221 | 221 | 264 | 270 | 323 | 326 | 140 | 140 | 188 | 196 | 200 | 200 | 309 | 309 |
| CT27 | 117 | 127 | 221 | 221 | 264 | 270 | 326 | 326 | 140 | 152 | 188 | 188 | 200 | 200 | 300 | 300 |
| CT28 | 117 | 127 | 221 | 221 | 264 | 270 | 323 | 326 | 140 | 140 | 188 | 196 | 200 | 200 | 309 | 309 |
| CT29 | 127 | 127 | 221 | 221 | 264 | 270 | 326 | 326 | 131 | 131 | 188 | 200 | 200 | 200 | 321 | 321 |
| CT30 | 117 | 117 | 221 | 221 | 264 | 270 | 326 | 332 | 152 | 152 | 188 | 192 | 200 | 200 | 309 | 309 |
| CT31 | 117 | 117 | 221 | 221 | 270 | 270 | 323 | 326 | 140 | 152 | 188 | 196 | 200 | 200 | 300 | 309 |
| CT32 | 117 | 117 | 221 | 221 | 264 | 270 | 323 | 326 | 140 | 152 | 188 | 188 | 200 | 200 | 309 | 309 |
| CT33 | 117 | 123 | 221 | 221 | 270 | 273 | 326 | 326 | 152 | 152 | 188 | 188 | 200 | 200 | 300 | 300 |
| CT34 | 117 | 117 | 221 | 221 | 270 | 270 | 323 | 326 | 140 | 152 | 188 | 196 | 200 | 200 | 300 | 309 |
| CT35 | 117 | 127 | 218 | 221 | 264 | 270 | 326 | 326 | 152 | 152 | 188 | 192 | 200 | 200 | 309 | 309 |
| CT36 | 117 | 127 | 221 | 221 | 264 | 273 | 326 | 326 | 131 | 152 | 188 | 196 | 200 | 200 | 300 | 321 |
| CT37 | 127 | 127 | 221 | 221 | 264 | 270 | 323 | 326 | 131 | 140 | 192 | 196 | 200 | 200 | 309 | 321 |
| CT38 | 117 | 117 | 221 | 221 | 276 | 279 | 323 | 332 | 140 | 152 | 188 | 192 | 200 | 200 | 309 | 309 |
| CT39 | 117 | 117 | 221 | 221 | 276 | 276 | 332 | 332 | 152 | 152 | 188 | 188 | 200 | 200 | 309 | 309 |
| CT40 | 117 | 127 | 221 | 221 | 264 | 273 | 326 | 326 | 131 | 152 | 188 | 196 | 200 | 200 | 300 | 321 |
| CZE187 | 117 | 117 | 221 | 221 | 270 | 270 | 329 | 329 | 146 | 152 | 192 | 192 | 200 | 200 | 303 | 303 |
| FRA189 | 103 | 117 | 218 | 218 | 264 | 270 | 332 | 332 | 134 | 146 | 192 | 192 | 200 | 200 | 294 | 303 |
| FRA190 | 113 | 113 | 218 | 221 | 264 | 270 | 320 | 332 | 146 | 152 | 192 | 192 | 200 | 200 | 306 | 306 |
| FRA193 | 117 | 125 | 221 | 221 | 264 | 270 | 332 | 332 | 134 | 146 | 192 | 192 | 200 | 200 | 318 | 336 |
| GER188 | 117 | 117 | 218 | 221 | 264 | 264 | 332 | 332 | 146 | 152 | 188 | 192 | 200 | 200 | 312 | 312 |
| GER195 | 117 | 117 | 218 | 218 | 264 | 270 | 329 | 332 | 128 | 146 | 192 | 192 | 200 | 200 | 303 | 303 |
| GER240 | 115 | 117 | 221 | 221 | 264 | 279 | 326 | 329 | 146 | 146 | 192 | 192 | 200 | 200 | 294 | 309 |
| GER91 | 103 | 117 | 221 | 221 | 279 | 279 | 320 | 320 | 146 | 152 | 192 | 192 | 200 | 200 | 321 | 321 |
| HA209 | 117 | 117 | 221 | 221 | 264 | 279 | 326 | 329 | 152 | 152 | 188 | 192 | 200 | 200 | 309 | 315 |
| HA210 | 117 | 127 | 221 | 221 | 264 | 279 | 326 | 326 | 152 | 152 | 188 | 192 | 200 | 200 | 309 | 315 |
| HA211 | 117 | 127 | 221 | 221 | 270 | 270 | 326 | 332 | 131 | 164 | 188 | 192 | 200 | 200 | 309 | 324 |
| HA77 | 117 | 112 | 218 | 221 | 264 | 270 | 326 | 329 | 137 | 164 | 188 | 192 | 200 | 200 | 297 | 300 |
| HA78 | 117 | 127 | 221 | 221 | 264 | 264 | 329 | 329 | 152 | 164 | 188 | 196 | 200 | 200 | 315 | 315 |
| HA79 | 117 | 123 | 221 | 221 | 264 | 264 | 326 | 326 | 152 | 164 | 188 | 188 | 200 | 200 | 300 | 300 |
| HA80 | 117 | 127 | 221 | 221 | 264 | 264 | 326 | 329 | 152 | 152 | 188 | 188 | 200 | 200 | 300 | 300 |
| HOL200 | 123 | 123 | 218 | 221 | 264 | 270 | 326 | 329 | 152 | 152 | 192 | 192 | 200 | 200 | 312 | 312 |
| HOL230 | 117 | 117 | 221 | 221 | 264 | 273 | 323 | 326 | 140 | 152 | 188 | 188 | 200 | 200 | 300 | 309 |
| HUN192 | 117 | 121 | 218 | 221 | 270 | 270 | 329 | 332 | 146 | 152 | 188 | 192 | 200 | 200 | 297 | 321 |
| HUN198 | 117 | 117 | 221 | 221 | 270 | 279 | 326 | 332 | 146 | 152 | 192 | 192 | 200 | 200 | 303 | 318 |
| HUN212 | 105 | 117 | 218 | 218 | 270 | 270 | 329 | 332 | 134 | 146 | 188 | 188 | 200 | 200 | 294 | 318 |
| HUN213 | 115 | 117 | 218 | 221 | 264 | 270 | 329 | 332 | 137 | 146 | 188 | 192 | 200 | 200 | 303 | 303 |
| HUN70 | 117 | 117 | 218 | 218 | 270 | 270 | 332 | 332 | 146 | 146 | 188 | 192 | 200 | 200 | 315 | 321 |
| HUN84 | 117 | 117 | 218 | 221 | 264 | 264 | 326 | 329 | 146 | 152 | 192 | 192 | 200 | 200 | 303 | 303 |
| HUN87 | 117 | 121 | 218 | 221 | 264 | 270 | 326 | 329 | 137 | 152 | 188 | 188 | 200 | 200 | 273 | 303 |
| HUN89 | 117 | 117 | 221 | 221 | 264 | 279 | 326 | 329 | 128 | 146 | 192 | 192 | 200 | 200 | 303 | 306 |
| IND179 | 123 | 123 | 221 | 221 | 270 | 276 | 323 | 329 | 140 | 152 | 192 | 196 | 200 | 200 | 303 | 303 |
| IND180 | 113 | 127 | 221 | 221 | 270 | 270 | 326 | 332 | 152 | 152 | 192 | 196 | 200 | 200 | 306 | 309 |
| IND207 | 121 | 123 | 218 | 221 | 270 | 270 | 323 | 323 | 152 | 152 | 192 | 196 | 200 | 200 | 309 | 309 |
| IND229 | 123 | 123 | 218 | 221 | 276 | 279 | 326 | 326 | 152 | 152 | 188 | 192 | 200 | 200 | 306 | 306 |
| IND86 | 117 | 117 | 218 | 221 | 279 | 279 | 326 | 326 | 152 | 164 | 192 | 192 | 200 | 200 | 309 | 309 |
| ITA191 | 117 | 117 | 218 | 221 | 270 | 270 | 317 | 329 | 146 | 152 | 192 | 192 | 200 | 200 | 297 | 306 |
| ITA194 | 121 | 121 | 218 | 218 | 270 | 270 | 332 | 332 | 134 | 143 | 188 | 192 | 200 | 200 | 300 | 318 |
| ITA88 | 103 | 117 | 218 | 218 | 264 | 270 | 320 | 329 | 146 | 152 | 192 | 192 | 200 | 200 | 306 | 306 |
| JAM236 | 117 | 117 | 221 | 221 | 270 | 279 | 329 | 329 | 164 | 164 | 188 | 192 | 200 | 200 | 300 | 300 |
| JAM237 | 117 | 117 | 221 | 221 | 264 | 270 | 329 | 329 | 164 | 164 | 188 | 188 | 200 | 200 | 309 | 309 |
| JAM65 | 117 | 123 | 218 | 218 | 270 | 276 | 320 | 326 | 152 | 164 | 196 | 200 | 200 | 221 | 300 | 321 |
| JAM66 | 127 | 127 | 218 | 221 | 270 | 270 | 326 | 329 | 152 | 164 | 188 | 200 | 200 | 200 | 309 | 309 |
| JAP196 | 113 | 113 | 218 | 218 | 270 | 270 | 326 | 326 | 143 | 146 | 196 | 196 | 200 | 200 | 306 | 306 |
| JAP241 | 109 | 123 | 221 | 221 | 270 | 270 | 320 | 320 | 128 | 128 | 192 | 200 | 200 | 200 | 306 | 306 |
| JAP242 | 103 | 109 | 221 | 221 | 270 | 270 | 317 | 326 | 128 | 143 | 192 | 200 | 200 | 200 | 300 | 306 |
| KOR186 | 109 | 113 | 218 | 221 | 270 | 270 | 320 | 326 | 128 | 146 | 192 | 200 | 200 | 200 | 321 | 321 |
| KOR204 | 113 | 123 | 218 | 221 | 270 | 270 | 320 | 326 | 134 | 146 | 192 | 196 | 200 | 200 | 297 | 297 |
| KOR248 | 113 | 117 | 221 | 221 | 270 | 270 | 326 | 329 | 128 | 128 | 192 | 196 | 200 | 200 | 297 | 306 |
| KOR249 | 113 | 123 | 218 | 221 | 270 | 270 | 326 | 326 | 131 | 137 | 192 | 192 | 200 | 200 | 294 | 294 |
| KURD214 | 119 | 119 | 221 | 221 | 264 | 264 | 326 | 326 | 128 | 152 | 192 | 192 | 200 | 200 | 294 | 294 |
| KURD215 | 117 | 123 | 221 | 221 | 264 | 264 | 326 | 326 | 152 | 152 | 192 | 192 | 200 | 200 | 306 | 306 |
| KY1 | 125 | 133 | 221 | 221 | 270 | 270 | 326 | 326 | 152 | 152 | 192 | 196 | 200 | 200 | 309 | 309 |
| KY165 | 117 | 117 | 221 | 221 | 270 | 270 | 326 | 326 | 152 | 152 | 188 | 196 | 200 | 200 | 309 | 309 |
| KY166 | 117 | 117 | 221 | 221 | 270 | 270 | 326 | 326 | 152 | 152 | 188 | 188 | 200 | 200 | 309 | 309 |
| KY167 | 117 | 127 | 221 | 221 | 270 | 270 | 326 | 329 | 152 | 152 | 188 | 188 | 200 | 200 | 309 | 321 |
| KY168 | 117 | 127 | 224 | 224 | 264 | 270 | 326 | 326 | 152 | 152 | 196 | 196 | 200 | 200 | 309 | 321 |
| KY169 | 117 | 127 | 218 | 221 | 264 | 270 | 323 | 326 | 152 | 152 | 192 | 192 | 200 | 200 | 309 | 309 |
| KY2 | 123 | 133 | 218 | 221 | 270 | 270 | 323 | 326 | 140 | 152 | 188 | 192 | 200 | 200 | 315 | 318 |
| KY25 | 121 | 133 | 218 | 221 | 270 | 270 | 323 | 326 | 152 | 152 | 196 | 196 | 221 | 221 | 306 | 312 |
| KY26 | 123 | 123 | 221 | 221 | 270 | 270 | 323 | 329 | 152 | 152 | 188 | 188 | 200 | 221 | 303 | 309 |
| KY27 | 123 | 123 | 218 | 221 | 270 | 270 | 323 | 326 | 152 | 152 | 192 | 192 | 200 | 221 | 303 | 309 |
| KY28 | 123 | 123 | 221 | 221 | 270 | 270 | 323 | 326 | 137 | 152 | 196 | 196 | 200 | 200 | 303 | 303 |
| KY29 | 113 | 127 | 218 | 221 | 270 | 270 | 323 | 326 | 137 | 152 | 192 | 192 | 221 | 221 | 306 | 312 |
| KY3 | 123 | 127 | 218 | 221 | 270 | 270 | 323 | 332 | 137 | 164 | 192 | 192 | 200 | 221 | 312 | 327 |
| KY30 | 117 | 123 | 218 | 221 | 270 | 270 | 326 | 329 | 137 | 137 | 188 | 196 | 200 | 221 | 309 | 309 |
| KY31 | 113 | 117 | 218 | 218 | 264 | 270 | 323 | 326 | 140 | 140 | 192 | 192 | 200 | 200 | 306 | 312 |
| KY32 | 119 | 127 | 218 | 221 | 264 | 264 | 326 | 326 | 152 | 152 | 196 | 196 | 200 | 200 | 309 | 309 |
| KY4 | 117 | 123 | 221 | 221 | 264 | 270 | 323 | 329 | 140 | 152 | 188 | 196 | 200 | 200 | 306 | 309 |
| KY49 | 123 | 123 | 221 | 221 | 264 | 270 | 323 | 332 | 140 | 152 | 188 | 188 | 200 | 200 | 312 | 312 |
| KY5 | 117 | 127 | 218 | 221 | 264 | 264 | 329 | 329 | 137 | 152 | 192 | 192 | 200 | 200 | 306 | 327 |
| KY50 | 117 | 123 | 221 | 221 | 270 | 270 | 329 | 329 | 152 | 164 | 188 | 188 | 200 | 200 | 303 | 318 |
| KY51 | 117 | 123 | 221 | 221 | 270 | 270 | 320 | 323 | 152 | 152 | 188 | 188 | 200 | 221 | 309 | 321 |
| KY52 | 117 | 117 | 218 | 218 | 264 | 270 | 323 | 329 | 137 | 152 | 192 | 192 | 200 | 200 | 303 | 315 |
| KY53 | 123 | 133 | 221 | 221 | 264 | 273 | 323 | 332 | 137 | 152 | 192 | 192 | 200 | 221 | 309 | 309 |
| KY54 | 117 | 127 | 221 | 221 | 270 | 270 | 326 | 332 | 140 | 152 | 192 | 196 | 200 | 200 | 309 | 318 |
| KY55 | 133 | 133 | 221 | 221 | 264 | 264 | 335 | 335 | 152 | 152 | 196 | 196 | 200 | 200 | 309 | 309 |
| KY56 | 135 | 135 | 221 | 221 | 264 | 264 | 326 | 335 | 152 | 152 | 188 | 188 | 200 | 200 | 303 | 312 |
| KY6 | 123 | 133 | 221 | 221 | 264 | 273 | 323 | 332 | 352 | 152 | 192 | 192 | 200 | 200 | 309 | 309 |
| KY7 | 117 | 127 | 221 | 221 | 270 | 279 | 323 | 326 | 152 | 152 | 188 | 192 | 200 | 200 | 312 | 312 |
| KY74 | 123 | 123 | 221 | 221 | 270 | 270 | 323 | 329 | 152 | 152 | 188 | 192 | 200 | 200 | 297 | 309 |
| KY75 | 117 | 117 | 218 | 221 | 264 | 273 | 323 | 326 | 152 | 152 | 192 | 196 | 200 | 200 | 309 | 309 |
| KY76 | 117 | 117 | 218 | 221 | 270 | 270 | 329 | 329 | 137 | 137 | 188 | 192 | 200 | 200 | 315 | 315 |
| KY8 | 123 | 129 | 218 | 218 | 270 | 270 | 326 | 326 | 152 | 152 | 188 | 188 | 200 | 200 | 303 | 312 |
| MEX233 | 117 | 121 | 218 | 218 | 264 | 264 | 320 | 329 | 137 | 152 | 192 | 192 | 200 | 200 | 318 | 318 |
| MEX246 | 117 | 117 | 221 | 221 | 264 | 264 | 323 | 323 | 152 | 164 | 188 | 188 | 200 | 221 | 309 | 309 |
| MEX57 | 117 | 117 | 218 | 221 | 264 | 270 | 323 | 323 | 131 | 152 | 188 | 192 | 200 | 200 | 309 | 309 |
| MEX58 | 117 | 117 | 221 | 221 | 264 | 264 | 326 | 329 | 152 | 164 | 188 | 192 | 200 | 200 | 309 | 309 |
| MEX59 | 113 | 117 | 218 | 218 | 270 | 270 | 329 | 332 | 152 | 164 | 192 | 192 | 221 | 221 | 288 | 315 |
| MEX60 | 117 | 117 | 218 | 221 | 264 | 279 | 323 | 329 | 164 | 164 | 188 | 188 | 200 | 200 | 309 | 315 |
| MEX85 | 117 | 117 | 218 | 221 | 264 | 270 | 323 | 329 | 152 | 164 | 188 | 188 | 200 | 221 | 309 | 315 |
| NEP221 | 123 | 123 | 218 | 218 | 270 | 270 | 317 | 323 | 152 | 152 | 188 | 192 | 200 | 200 | 288 | 318 |
| NIG222 | 123 | 123 | 218 | 218 | 264 | 270 | 323 | 326 | 134 | 155 | 192 | 192 | 200 | 200 | 309 | 309 |
| OR93 | 117 | 117 | 221 | 221 | 264 | 270 | 326 | 332 | 140 | 152 | 188 | 188 | 200 | 200 | 300 | 309 |
| OR94 | 117 | 117 | 221 | 221 | 264 | 270 | 326 | 332 | 140 | 152 | 188 | 188 | 200 | 200 | 300 | 309 |
| PAK226 | 115 | 127 | 218 | 221 | 264 | 270 | 326 | 332 | 152 | 152 | 192 | 196 | 200 | 200 | 300 | 300 |
| PAK227 | 117 | 127 | 218 | 218 | 264 | 264 | 326 | 326 | 152 | 152 | 188 | 196 | 200 | 200 | 300 | 300 |
| POL216 | 117 | 117 | 218 | 221 | 270 | 270 | 332 | 332 | 146 | 146 | 188 | 188 | 200 | 200 | 300 | 318 |
| POL228 | 121 | 123 | 221 | 221 | 264 | 270 | 332 | 332 | 134 | 152 | 192 | 192 | 200 | 200 | 297 | 297 |
| POL71 | 121 | 121 | 221 | 221 | 270 | 270 | 332 | 332 | 131 | 152 | 192 | 192 | 200 | 200 | 303 | 303 |
| ROM203 | 117 | 117 | 218 | 218 | 264 | 270 | 320 | 320 | 137 | 146 | 188 | 192 | 200 | 200 | 321 | 321 |
| RUS197 | 117 | 121 | 218 | 218 | 270 | 270 | 329 | 332 | 146 | 146 | 192 | 192 | 200 | 200 | 300 | 303 |
| RUS205 | 117 | 117 | 218 | 218 | 270 | 270 | 326 | 332 | 152 | 152 | 192 | 192 | 200 | 200 | 312 | 318 |
| RUS206 | 117 | 117 | 218 | 218 | 270 | 270 | 326 | 332 | 137 | 140 | 192 | 192 | 200 | 200 | 300 | 312 |
| RUS90 | 121 | 125 | 218 | 218 | 270 | 270 | 326 | 329 | 146 | 152 | 192 | 192 | 200 | 200 | 297 | 315 |
| SAF208 | 115 | 115 | 221 | 221 | 276 | 276 | 326 | 335 | 128 | 152 | 192 | 192 | 200 | 200 | 309 | 309 |
| SAF220 | 117 | 117 | 218 | 218 | 264 | 264 | 323 | 323 | 152 | 152 | 192 | 192 | 200 | 200 | 309 | 309 |
| SAF247 | 123 | 123 | 221 | 221 | 264 | 264 | 323 | 323 | 152 | 155 | 192 | 192 | 200 | 200 | 309 | 309 |
| SAF250 | 117 | 117 | 221 | 221 | 264 | 264 | 323 | 323 | 152 | 152 | 192 | 192 | 200 | 200 | 309 | 309 |
| SAF251 | 117 | 123 | 218 | 218 | 264 | 264 | 323 | 326 | 152 | 152 | 192 | 192 | 200 | 200 | 309 | 309 |
| SAF252 | 117 | 217 | 221 | 221 | 264 | 264 | 323 | 329 | 152 | 155 | 192 | 192 | 200 | 200 | 309 | 309 |
| SLe234 | 123 | 123 | 218 | 218 | 270 | 270 | 323 | 326 | 152 | 155 | 192 | 192 | 200 | 200 | 309 | 309 |
| SLe235 | 123 | 123 | 218 | 218 | 270 | 270 | 326 | 326 | 152 | 152 | 192 | 192 | 200 | 200 | 309 | 309 |
| SPA202 | 119 | 123 | 221 | 221 | 270 | 270 | 326 | 329 | 128 | 143 | 192 | 192 | 200 | 200 | 306 | 306 |
| SPA92 | 115 | 115 | 221 | 221 | 264 | 276 | 329 | 332 | 152 | 152 | 192 | 192 | 200 | 200 | 303 | 303 |
| THI232 | 123 | 123 | 221 | 221 | 270 | 270 | 326 | 326 | 152 | 152 | 192 | 192 | 200 | 200 | 300 | 309 |
| TN10 | 117 | 123 | 221 | 221 | 270 | 270 | 320 | 323 | 152 | 152 | 192 | 192 | 200 | 221 | 303 | 309 |
| TN105 | 123 | 123 | 218 | 221 | 270 | 279 | 323 | 323 | 128 | 128 | 188 | 192 | 200 | 200 | 309 | 309 |
| TN106 | 123 | 123 | 218 | 223 | 270 | 279 | 323 | 323 | 128 | 128 | 188 | 192 | 200 | 200 | 309 | 309 |
| TN107 | 127 | 127 | 221 | 221 | 264 | 264 | 326 | 326 | 152 | 152 | 188 | 188 | 200 | 200 | 303 | 303 |
| TN108 | 117 | 117 | 221 | 221 | 264 | 270 | 326 | 329 | 152 | 152 | 188 | 188 | 200 | 200 | 303 | 312 |
| TN109 | 117 | 117 | 221 | 221 | 264 | 276 | 323 | 329 | 128 | 146 | 188 | 192 | 200 | 200 | 309 | 309 |
| TN11 | 117 | 127 | 221 | 221 | 264 | 276 | 326 | 332 | 152 | 164 | 192 | 192 | 200 | 221 | 309 | 309 |
| TN110 | 117 | 127 | 218 | 221 | 264 | 270 | 326 | 332 | 152 | 164 | 192 | 200 | 200 | 200 | 309 | 315 |
| TN111 | 117 | 127 | 218 | 221 | 264 | 270 | 326 | 332 | 152 | 164 | 192 | 200 | 200 | 200 | 309 | 315 |
| TN112 | 115 | 123 | 221 | 221 | 264 | 264 | 329 | 329 | 152 | 152 | 188 | 192 | 200 | 200 | 300 | 321 |
| TN113 | 117 | 127 | 218 | 221 | 270 | 270 | 326 | 329 | 152 | 152 | 192 | 192 | 200 | 200 | 309 | 309 |
| TN114 | 117 | 117 | 218 | 218 | 270 | 270 | 326 | 329 | 152 | 152 | 188 | 188 | 200 | 200 | 300 | 303 |
| TN115 | 117 | 117 | 221 | 221 | 264 | 270 | 317 | 323 | 152 | 152 | 192 | 192 | 221 | 221 | 297 | 309 |
| TN116 | 117 | 123 | 221 | 221 | 270 | 279 | 323 | 326 | 164 | 164 | 188 | 192 | 200 | 200 | 309 | 309 |
| TN117 | 117 | 117 | 221 | 221 | 270 | 270 | 323 | 326 | 152 | 152 | 188 | 188 | 200 | 200 | 309 | 309 |
| TN118 | 117 | 117 | 218 | 221 | 270 | 273 | 326 | 326 | 152 | 152 | 188 | 188 | 200 | 200 | 300 | 309 |
| TN119 | 117 | 117 | 218 | 221 | 273 | 276 | 329 | 329 | 152 | 152 | 188 | 200 | 200 | 200 | 315 | 315 |
| TN12 | 117 | 127 | 221 | 221 | 264 | 270 | 326 | 326 | 152 | 152 | 192 | 192 | 200 | 200 | 318 | 318 |
| TN120 | 117 | 117 | 218 | 218 | 273 | 273 | 329 | 329 | 152 | 152 | 188 | 200 | 200 | 200 | 300 | 315 |
| TN13 | 117 | 127 | 221 | 221 | 264 | 270 | 326 | 326 | 152 | 152 | 188 | 192 | 200 | 221 | 309 | 318 |
| TN14 | 117 | 125 | 221 | 221 | 264 | 264 | 326 | 332 | 137 | 137 | 200 | 200 | 200 | 200 | 294 | 318 |
| TN15 | 117 | 123 | 221 | 221 | 270 | 273 | 329 | 329 | 128 | 152 | 188 | 192 | 200 | 200 | 312 | 315 |
| TN16 | 117 | 117 | 218 | 221 | 270 | 279 | 329 | 332 | 137 | 137 | 188 | 188 | 200 | 200 | 300 | 315 |
| TN41 | 115 | 127 | 218 | 221 | 270 | 270 | 320 | 326 | 140 | 152 | 196 | 196 | 200 | 200 | 312 | 324 |
| TN42 | 115 | 127 | 218 | 221 | 270 | 270 | 320 | 326 | 140 | 152 | 196 | 196 | 200 | 200 | 309 | 321 |
| TN43 | 113 | 117 | 221 | 221 | 264 | 270 | 320 | 326 | 140 | 155 | 188 | 196 | 200 | 200 | 309 | 309 |
| TN44 | 127 | 127 | 221 | 221 | 264 | 270 | 320 | 326 | 140 | 155 | 196 | 196 | 200 | 200 | 309 | 321 |
| TN45 | 117 | 117 | 218 | 221 | 264 | 279 | 323 | 329 | 152 | 152 | 188 | 188 | 200 | 221 | 300 | 309 |
| TN46 | 117 | 127 | 221 | 221 | 264 | 270 | 326 | 329 | 152 | 152 | 196 | 196 | 200 | 200 | 309 | 321 |
| TN47 | 127 | 127 | 221 | 221 | 264 | 264 | 326 | 329 | 140 | 164 | 192 | 192 | 200 | 200 | 303 | 309 |
| TN48 | 117 | 127 | 221 | 221 | 270 | 270 | 323 | 326 | 137 | 137 | 188 | 188 | 200 | 200 | 300 | 309 |
| TN9 | 117 | 127 | 221 | 221 | 264 | 270 | 323 | 329 | 152 | 152 | 192 | 192 | 200 | 221 | 315 | 315 |
| TUR199 | 113 | 117 | 218 | 221 | 264 | 264 | 326 | 326 | 134 | 152 | 192 | 192 | 200 | 200 | 309 | 312 |
| TUR253 | 103 | 117 | 218 | 221 | 270 | 270 | 326 | 329 | 146 | 152 | 188 | 188 | 200 | 200 | 303 | 312 |
| TUR254 | 115 | 117 | 218 | 218 | 270 | 270 | 326 | 329 | 152 | 152 | 188 | 192 | 200 | 200 | 303 | 309 |
| UGA238 | 115 | 115 | 218 | 218 | 264 | 264 | 326 | 326 | 152 | 152 | 192 | 192 | 200 | 200 | 309 | 309 |
| UGA239 | 115 | 115 | 218 | 218 | 264 | 264 | 326 | 326 | 152 | 152 | 192 | 192 | 200 | 200 | 309 | 309 |
| UZB255 | 115 | 117 | 218 | 218 | 270 | 270 | 332 | 332 | 137 | 137 | 192 | 192 | 200 | 200 | 300 | 300 |
| UZB256 | 123 | 123 | 218 | 221 | 264 | 270 | 323 | 326 | 137 | 152 | 192 | 196 | 200 | 200 | 306 | 306 |
| WV151 | 113 | 117 | 221 | 221 | 270 | 270 | 323 | 326 | 152 | 164 | 192 | 196 | 221 | 221 | 300 | 309 |
| WV152 | 117 | 117 | 218 | 221 | 270 | 270 | 323 | 323 | 137 | 152 | 192 | 192 | 200 | 200 | 318 | 318 |
| WV153 | 127 | 127 | 218 | 221 | 264 | 264 | 326 | 329 | 152 | 152 | 188 | 192 | 200 | 221 | 315 | 321 |
| WV154 | 123 | 127 | 218 | 221 | 264 | 264 | 323 | 329 | 152 | 152 | 192 | 196 | 200 | 200 | 309 | 309 |
| WV155 | 123 | 127 | 221 | 221 | 264 | 264 | 326 | 326 | 143 | 164 | 188 | 192 | 200 | 221 | 309 | 309 |
| WV156 | 123 | 127 | 221 | 221 | 264 | 264 | 326 | 326 | 143 | 164 | 188 | 192 | 200 | 221 | 309 | 309 |
| WV157 | 117 | 123 | 218 | 221 | 270 | 270 | 329 | 332 | 152 | 152 | 188 | 192 | 200 | 200 | 309 | 315 |
| WV158 | 117 | 127 | 221 | 221 | 270 | 270 | 320 | 326 | 152 | 152 | 192 | 192 | 200 | 200 | 309 | 321 |
| WV159 | 117 | 127 | 218 | 221 | 270 | 270 | 326 | 326 | 131 | 152 | 192 | 192 | 200 | 200 | 309 | 312 |
| WV160 | 123 | 127 | 218 | 218 | 270 | 270 | 323 | 332 | 152 | 152 | 192 | 192 | 200 | 200 | 309 | 309 |
| WV161 | 123 | 127 | 218 | 218 | 270 | 270 | 323 | 326 | 131 | 131 | 192 | 196 | 200 | 221 | 309 | 309 |
| WV162 | 117 | 123 | 218 | 221 | 264 | 270 | 323 | 326 | 137 | 140 | 192 | 192 | 200 | 200 | 309 | 315 |
| WV163 | 123 | 123 | 221 | 221 | 270 | 270 | 323 | 326 | 137 | 140 | 188 | 192 | 200 | 200 | 309 | 315 |
| WV164 | 123 | 127 | 218 | 218 | 270 | 270 | 326 | 332 | 140 | 152 | 192 | 200 | 200 | 200 | 306 | 309 |
| WV17 | 125 | 125 | 221 | 221 | 270 | 270 | 326 | 329 | 146 | 152 | 188 | 196 | 200 | 200 | 309 | 321 |
| WV18 | 117 | 117 | 221 | 221 | 264 | 270 | 326 | 326 | 140 | 152 | 196 | 196 | 200 | 200 | 309 | 303 |
| WV19 | 117 | 117 | 221 | 221 | 264 | 270 | 323 | 329 | 152 | 164 | 192 | 192 | 200 | 200 | 309 | 315 |
| WV20 | 127 | 127 | 218 | 218 | 270 | 270 | 326 | 326 | 131 | 131 | 192 | 192 | 200 | 200 | 309 | 309 |
| WV21 | 117 | 123 | 221 | 221 | 270 | 276 | 326 | 326 | 140 | 140 | 188 | 196 | 200 | 200 | 309 | 315 |
| WV22 | 117 | 123 | 221 | 221 | 270 | 270 | 323 | 326 | 140 | 140 | 188 | 196 | 200 | 200 | 309 | 309 |
| WV23 | 117 | 127 | 221 | 221 | 270 | 270 | 323 | 323 | 152 | 152 | 188 | 192 | 200 | 200 | 309 | 315 |
| WV24 | 127 | 127 | 221 | 221 | 264 | 276 | 326 | 326 | 152 | 152 | 192 | 192 | 200 | 200 | 309 | 312 |
| WV33 | 117 | 117 | 221 | 221 | 270 | 270 | 329 | 329 | 152 | 152 | 192 | 192 | 200 | 200 | 309 | 315 |
| WV34 | 123 | 123 | 221 | 221 | 264 | 270 | 323 | 326 | 155 | 155 | 188 | 188 | 200 | 221 | 309 | 321 |
| WV35 | 117 | 123 | 218 | 218 | 270 | 270 | 326 | 332 | 152 | 152 | 196 | 200 | 200 | 200 | 309 | 309 |
| WV36 | 123 | 123 | 218 | 221 | 264 | 270 | 323 | 326 | 152 | 152 | 192 | 196 | 200 | 221 | 309 | 312 |
| WV37 | 117 | 123 | 218 | 221 | 270 | 279 | 326 | 326 | 137 | 137 | 188 | 192 | 200 | 221 | 315 | 321 |
| WV38 | 127 | 127 | 218 | 218 | 270 | 270 | 326 | 326 | 131 | 131 | 192 | 192 | 200 | 200 | 309 | 309 |
| WV39 | 117 | 123 | 221 | 221 | 264 | 270 | 329 | 329 | 152 | 164 | 188 | 192 | 200 | 221 | 309 | 309 |
| WV40 | 117 | 127 | 218 | 221 | 270 | 270 | 323 | 326 | 152 | 152 | 192 | 196 | 200 | 200 | 309 | 309 |
| WV95 | 117 | 117 | 221 | 221 | 270 | 270 | 323 | 332 | 164 | 164 | 192 | 196 | 200 | 200 | 309 | 315 |
| WV96 | 117 | 123 | 218 | 221 | 264 | 270 | 329 | 332 | 146 | 152 | 192 | 192 | 200 | 200 | 309 | 315 |
| ZIM244 | 117 | 123 | 218 | 218 | 264 | 270 | 323 | 326 | 152 | 155 | 192 | 192 | 200 | 200 | 309 | 309 |
| ZIM245 | 123 | 123 | 218 | 218 | 264 | 270 | 326 | 326 | 152 | 152 | 192 | 192 | 200 | 200 | 309 | 309 |

LEGEND

| | |
|---|---|
| AFG | Afghanistan |
| AK | Alaska, USA |
| AZ | Arizona, USA |
| CA | California, USA |
| CAM | Cambodia |
| CAN | Canada |
| CHI | China |
| COL | Colombia |
| CoR | Costa Rica |
| CT | Connecticut, USA |
| CZE | Czechoslovakia |
| FRA | France |
| GER | Germany |
| HA | Hawaii, USA |
| HOL | Holland |
| HUN | Hungary |
| IND | India |
| ITA | Italy |
| JAM | Jamaica |
| JAP | Japan |
| KOR | Korea |
| KURD | Kurdistan |
| KY | Kentucky, USA |
| MEX | Mexico |
| NEP | Nepal |
| NIG | Nigeria |
| OR | Oregon, USA |
| PAK | Pakistan |
| POL | Poland |
| ROM | Romania |
| RUS | Russia |
| SAF | South Africa |
| SLe | Sierra Leone |
| SPA | Spain |
| THI | Thailand |
| TN | Tennessee, USA |
| TUR | Turkey |
| UGA | Uganda |
| UZB | Uzbekistan |
| WV | West Virginia, USA |
| ZIM | Zimbabwe |

### REFERENCES

[1] G. S. Chopra, Man and marijuana, Int. J. Addiction, 4 (2) (1969) 215-247.
[2] R. E. Schultes, Man and marijuana, Nat. Hist. 82 (7) (1973) 58-63, 80, 82.
[3] R. Adams, Marihuana, Science 92 (1940) 115-119.
[4] E. Small, H. D. Beckstead, A. Chan, The evolution of cannabinoid phenotypes in Cannabis, Econ. Bot. 29 (1975) 219-232.
[5] W. A. Emboden, Jr., Ritual use of Cannabis sativa L.: a historical-ethnographic survey, in: P. T. Furst (Eds.), Flesh of the gods: the ritual use of hallucinogens, Praeger Publishers, NY and WA, 1972, pp. 214-235.
[6] E. Small, A. Cronquist, A practical and natural taxonomy for Cannabis, Taxon 25 (4)(1976) 405-435.
[7] R. Gillan, M. D. Cole, A. Linacre, J. W. Thorpe, N. D. Watson, Comparison of Cannabis sativa by random amplification of polymorphic DNA (RAPD) and HPLC of cannabinoids; a preliminary study, Sci. Justice 35 (3) (1995) 169-177.
[8] G. Siniscalco Gigliano, P. Caputo, S. Cozzolino, Ribosomal DNA analysis as a tool to identify specimens of Cannabis sativa L. of forensic interest, Sci. Justice 37 (3) (1997) 171-174.
[9] G. Siniscalco Gigliano, Identification of Cannabis sativa L. (Cannabaceae) using restriction profiles of the Internal Transcribed Spacer II (ITS2), Sci. Justice 38 (4) (1998) 225-230.
[10] G. Siniscalco Gigliano, Preliminary data on the usefulness of Internal Transcribed Spacer I (ITS1) sequence in Cannabis sativa L. identification, J. Forensic Sci. 44 (3) (1999) 475-477.
[11] M. Kohjyouma, I. J. Lee, O. Iida, K. Kurihara, K. Yamada Y. Makino, S. Sekita, M. Satake, Intraspecific variation in Cannabis sativa L. based on intergenic spacer region of chloroplast DNA, Biol. Pharm. Bull. 23 (6) (2000) 727-730.
[12] S. Gilmore, R. Peakall, J. Robertson, Short tandem repeat (STR) DNA markers are hypervariable and informative in Cannabis sativa: implications for forensic investigations, Forensic Sci. Int. 131 (2003) 65-74.
[13] H. M. Hsieh, R. J. Hou, L. C. Tsai, C. S. Wei, S. W. Liu, L. H. Huang, Y. C. Kuo, A. Linacre, J. C. I. Lee, A highly polymorphic STR locus in Cannabis sativa, Forensic Sci. Int. 131 (2003) 53-58.
[14] D. Balding, Forensic applications of microsatellite markers, in D. B. Goldstein and C. Schlötterer (eds.), Microsatellites Evolution and Applications, (1999) Oxford University Press, New York, pp. 198-210.
[15] K. Weising, H. Nybom, K. Wolff, M. Wieland, DNA fingerprinting in plants and fungi, Boca Ration Fla.: CRC Press (1995) 51-53.
[16] S. H. Li, H. Yi-Jiun, J. L. Brown, Isolation of tetranucleotide microsatellites from the Mexican jay Aphelocoma ulltramarina, Mol. Ecol. 6 (1997) 499-501.
[17] T. Pearson, Polygyny and extra-pair paternity in a population of southwestern willoflycatchers (Empidonax traillii extimus), Northern Arizona University, M.S. thesis (2002)
[18] L. Cardle, L. Ramsay, D. Milbourne, M. Macaulay, D. Marshall, R. Waugh, Computational and experimental characterization of physically clustered simple sequence repeats in plants, Genetics 156 (2000) 847-854.
[19] S. D. E. Park, The Excel Microsatellite Toolkit; Trypanotolerance in West African Cattle and the Population Genetic Effects of Selection, University of Dublin, Ph.D. thesis (2001).
[20] Anonymous, Microsat, the microsatellite distance program, Human population genetics laboratory, Department of genetics, Stanford, Calif., USA, (2001).
[21] J. Flesenstein, PHYLIP (Phylogeny Inference Package) version 3.5c, Seattle, Wash., USA, (1993).
[22] L. Excoffier, P. E. Smouse, J. M. Quattro, Analysis of molecular variance inferred from metric distances among DNA haplotypes: application to human mitochondrial DNA restriction data, Genetics 131 (1992) 179-191.
[23] R. Peakall, P. E. Smouse, GenAlEx V5: Genetic Analysis in Excel. Population genetic software for teaching and research. Australian National University, Canberra, Australia (2001) www.anu.edu.au/BoZo/GenAlEx/
[24] M. Perez-Reyes, W. R. White, S. A. McDonald, R. E. Hicks, A. R. Jeffcoat, and C. E. Cook, The pharmacological effects of daily marijuana smoking in humans, Pharmacol. Biochem. Behav. 40 (1991) 691-694.
[25] C. Kimpton, D. Fisher, S. Watson, M. Adams, A. Urquhart, J. Lygo, P. Gill. Evaluation of an automated DNA profiling system employing multiplex amplification of four tetrameric STR loci, Int. J. Legal Med. 106 (1994) 302-311.
[26] D. Watts, Genotyping STR loci using an automated DNA sequencer: in P. J. Lincoln, J. Thompson (Eds.), Forensic DNA Profiling Protocols, Humana Press, Totowa, N.J., 1998, pp. 193-208.
[27] H. Miller Coyle, A. B. Harper, R. A. Bever, M. A. Sanger, J. Germano-Presby, K. N. Zinnamon, M. A. Menotti-Raymond, V. A. David, Non-human typing: methods and casework applications, Proceedings of the American Academy of Forensic Sciences 55^{th} Annual Meeting, (2003) Feb. 17-22, Chicago, Ill., USA. p. 20.
[28] H. Miller Coyle, C. Ladd, T. Palmbach, H. C. Lee, The green revolution: botanical contributions to forensics and drug enforcement, Croatian Med. J. 42 (3) (2001) 340-345.

We claim:

1. An isolated nucleic acid comprising at least 12 consecutive nucleotides of a nucleotide sequence selected from the group consisting of SEQ ID NO: 1; complementary sequence of SEQ ID NO 1, SEQ ID NO: 2, complementary sequence of SEQ ID NO 2; SEQ ID NO: 3; complementary sequence of SEQ ID NO. 3; SEQ ID NO: 4; complementary sequence of SEQ ID NO: 4; SEQ ID NO: 5; complementary sequence of SEQ ID NO: 5; SEQ ID NO: 6; complementary sequence of SEQ ID NO. 6; SEQ ID NO: 7; complementary sequence of SEQ ID NO 7; SEQ ID NO: 8; complementary sequence of SEQ ID NO. 8; SEQ ID NO: 9; complementary sequence of SEQ ID NO: 9; SEQ ID NO: 10; complementary sequence of SEQ ID NO: 10; SEQ ID NO: 11; complementary sequence of SEQ ID NO: 11; SEQ ID NO: 12; complementary sequence of SEQ ID NO: 12; SEQ ID NO: 13; complementary sequence of SEQ ID NO: 13; SEQ ID NO: 14; complementary sequence of SEQ ID NO: 14; SEQ ID NO: 15; complementary sequence of SEQ ID NO: 15; SEQ ID NO: 16; complementary sequence of SEQ ID NO: 16; SEQ ID NO: 17; complementary sequence of SEQ ID NO: 17; SEQ ID NO: 18; complementary sequence of SEQ ID NO: 18; SEQ ID NO: 19; complementary sequence of SEQ ID NO: 19; SEQ ID NO: 20; complementary sequence of SEQ ID NO: 20; SEQ ID NO: 21; complementary sequence of SEQ ID NO: 21; SEQ ID NO: 22; complementary sequence of SEQ ID NO: 22; SEQ ID NO: 23; complementary sequence of SEQ ID NO: 23; SEQ ID NO: 24; complementary sequence of SEQ ID NO: 24; SEQ ID NO: 25; complementary sequence of SEQ ID NO: 25; SEQ ID NO: 26; complementary sequence of SEQ ID NO: 26; SEQ ID NO: 27; complementary sequence of SEQ ID NO: 27; SEQ ID NO: 28; and complementary sequence of SEQ ID NO: 28.

2. The isolated nucleic acid of claim 1, wherein the nucleic acid comprises at least 15 consecutive nucleotides of the nucleotide sequence.

3. The isolated nucleic acid of claim 1, wherein the nucleic acid comprises at least 18 consecutive nucleotides of the nucleotide sequence.

4. The isolated nucleic acid of claim 1 immobilized on a solid surface.

5. The isolated nucleic acid of claim 1, wherein the nucleic acid is capable of detecting Cannabis sativa L.

6. The isolated nucleic acid of claim 1, wherein the isolated nucleic acid is capable of being used in a multiplex cocktail for amplification of a STR from Cannabis sativa L.

7. A pair of forward and reverse primers for amplification of a STR located in DNA isolated from Cannabis sativa L., said pair being selected from the group consisting of SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 13 and SEQ ID NO: 14; SEQ ID NO:15 and SEQ ID NO: 16; and SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; and SEQ ID NO: 27 and SEQ ID NO:28.

8. The pair of forward and reverse primers of claims 7, wherein a member of said pair comprises an observable marker.

9. The pair of forward and reverse primers of claim 8, wherein said marker is a fluorescent label.

10. The pair of forward and reverse primers of claim 8, wherein said marker is a radioactive group.

11. The pair of forward and reverse primers of claim 7 as PCR primers in the detection of a Cannabis sativa L. species.

12. The pair of forward and reverse primers of claim 7, wherein said pair is capable of being used in a multiplex cocktail for amplification of STR from Cannabis sativa L.

13. A method for detecting a Cannabis sativa L. species in a sample comprising the steps of:
i. obtaining DNA from the sample,
ii. amplifying a STR marker loci in said DNA with a multiplex cocktail of claim 7 to form amplification products of various sizes and labels; and
iii. separating amplification products by size and primer label;
iv. scoring the results of said separation; and
v. comparing said scored results to analysis of DNA from a known species.

14. A method of linking a marijuana sample to a plant source comprising the steps of:
i. determining the identity of DNA in said sample by the method of claim 13;
ii. determining the identity of DNA in a sample from a plant by the method of claim 13; and
iii. comparing the identities of both samples to determine similarities.

15. A kit for use in the detection of a Cannabis sativa L. species by multiplex cocktail comprising a primer pair of claim 7.

16. The kit of claim 15, further comprising nucleic acids, enzymes and buffers suitable for causing amplification of STR in DNA from said species in a multiplex PCR instrument.

17. The kit of claim 15 detecting a Cannabis sativa L. species comprising:
i. a multiplex cocktail of claim 12;
ii. nucleic acids having an observable marker;
iii. a transcriptase; and
iv. buffers and salts suitable for causing polymerization of STR in DNA from said Cannabis sativa L. species in a PCR multiplex instrument.

18. The kit of claim 15, further comprising a control sample of DNA.
